(19) United States
(12) Patent Application Publication
(10) Pub. No.: US 2018/0325490 A1
(43) Pub. Date: Nov. 15, 2018

(54) PROBE DEVICE AND CONTROL METHOD THEREFOR

(71) Applicant: SAMSUNG ELECTRONICS CO., LTD., Suwon-si (KR)

(72) Inventors: Yeon-woo KU, Suwon-si (KR); Nam-seok CHANG, Hwaseong-si (KR); Young-hwan KIM, Hwaseong-si (KR); Nam-joo PARK, Suwon-si (KR); Jeong-su LEE, Suwon-si (KR); Seung-ho JANG, Incheon (KR)

(73) Assignee: SAMSUNG ELECTRONICS CO., LTD., Suwon-si (KR)

(21) Appl. No.: 15/774,765

(22) PCT Filed: Nov. 9, 2016
(86) PCT No.: PCT/KR2016/012860

§ 371 Date: May 9, 2018

(30) Foreign Application Priority Data
Nov. 9, 2015 (KR) 10-2015-0156525

(51) Int. Cl. A61B 8/00; A61B 8/06; A61B 8/08; A61N 7/02

(52) U.S. Cl. CPC A61B 8/4483; A61B 8/06; A61B 8/463; A61N 2007/0052; A61B 8/5207; A61B 8/485; A61N 7/02; A61B 8/488

(57) ABSTRACT

A probe device is disclosed. The probe device comprises: a matrix array analog front end (AFE) comprising a plurality of cells and outputting an electrical signal corresponding to each of the plurality of cells; a transducer unit for transducing, into an ultrasound signal, the electrical signal outputted from each of the plurality of cells; and a processor for grouping the plurality of cells into at least one group corresponding to at least one diagnosis mode, and performing control such that cells corresponding to each group outputs, through the transducer unit, an ultrasound signal having a characteristic differing according to a corresponding diagnosis mode. Therefore, various functions can be supported while using one probe device.

### TECHNICAL FIELD

The present disclosure relates to a probe device and a control method therefor, and more particularly, to a probe device including a matrix array analog front end (AFE) and a control method therefor.

### BACKGROUND ART

Thanks to the development of electronic technology, various kinds of electronic products are developing and are being distributed. In particular, various display apparatuses such as televisions (TVs), mobile phones, personal computers (PCs), notebook PCs, personal digital assistant (PDAs) are increasingly used in general households. The development of electronic technology influences the medical and health care fields.

In particular, many ultrasound examinations are conducted in the medical and health care fields. When such an ultrasound examination is conducted, a probe device is used to obtain an ultrasound image by transmitting and receiving ultrasound waves in contact with a patient's body.

However, a related-art ultrasound examination system has a necessary probe device fixed according to a function supported thereby, and thus there is inconvenience that, if a user wishes to add or change the function, the probe device should be replaced. For example, as shown in FIG. 1, the related-art ultrasound examination system may require a plurality of transducers according to diagnosis targets, and a plurality of probe devices including hardware corresponding to the transducers, and also, when a high intensity focused ultrasound (HIFU) pulse or an elastic wave pulse is used, the ultrasound examination system may require another necessary probe device to support these pulses.

Therefore, there is an increasing demand for a method for supporting various functions simply by using a single probe device.

### Detailed Description of the Present Disclosure

### Technical Objects

The present disclosure has been developed in order to solve the above-mentioned problems, and an object of the present disclosure is to provide a probe device which can reconfigure a plurality of cells included in a matrix array analog front end (AFE), and control to output an ultrasound signal having a different characteristic according to a corresponding diagnosis mode, and a control method therefor.

### Technical Solving Method

According to an embodiment of the present disclosure to achieve the above-described object, a probe device including: a matrix array analog front end (AFE) including a plurality of cells, and configured to output an electric signal corresponding to each of the plurality of cells; a transducer unit configured to transduce the electric signal outputted from each of the plurality of cells into an ultrasound signal; and a processor configured to group the plurality of cells into at least one group corresponding to at least one diagnosis mode, and to control cells corresponding to each group to output an ultrasound signal having a different characteristic according to a corresponding diagnosis mode through the transducer unit.

Herein, the processor may be configured to transmit the ultrasound signal having the different characteristic to a target, and to generate a different image based on an ultrasound signal received from the target, or to transmit a focused ultrasound signal to the target.

In addition, the processor may be configured to control cells corresponding to each diagnosis mode to adjust at least one of a size, a frequency, and a focusing point of the ultrasound signal, and to output a different ultrasound signal.

In addition, the diagnosis mode may include a first mode for generating a second harmonic image, a second mode for generating a plurality of images related to a plurality of targets, a third mode for transmitting a focused ultrasound signal, and a fourth mode for transmitting a high-voltage/low-voltage ultrasound signal.

In addition, in the first mode, the processor may be configured to divide a plurality of cells belonging to a group corresponding to the first mode into a first transmission and reception region and a second transmission and reception region, to transmit a first ultrasound signal and a second ultrasound signal having a phase difference of 180 degrees to a target, simultaneously, through the first transmission and reception region and the second transmission and reception region, and to generate the second harmonic image based on the first ultrasound signal and the second ultrasound signal received from the target.

In addition, in the second mode, the processor may be configured to divide a plurality of cells belonging to a group corresponding to the second mode into a plurality of transmission and reception regions to transmit and receive the ultrasound signal to and from the plurality of targets, and to detect an abnormal portion by comparing images generated from the plurality of transmission and reception regions.

In addition, in the third mode, the processor may be configured to divide a plurality of cells belonging to a group corresponding to the third group into a first transmission region and a second transmission and reception region, to transmit a focused ultrasound signal for a treatment to a target through the first transmission region, to transmit and receive the ultrasound signal to and from the target through the second transmission and reception region, and to generate an image regarding a progress of a treatment by the focused ultrasound signal.

In addition, in the fourth mode, the processor may be configured to divide a plurality of cells belonging to a group corresponding to the fourth mode into a first transmission and reception region for generating and transmitting and receiving a high-voltage ultrasound signal, and a second transmission and reception region for generating and transmitting and receiving a low-voltage ultrasound signal, to generate a first image based on the transmitted and received high-voltage ultrasound signal, and to generate a second image based on the transmitted and received low-voltage ultrasound signal.

In addition, the processor may be configured to perform beamforming with respect to the plurality of cells belonging to each group, based on at least one of a depth, a size, and a location of a target, such that an ultrasound signal outputted through the transducer unit is focused onto the target.

In addition, the probe device may further include a display, and the processor may be configured to display the generated image through the display.

In addition, the probe device may further include a communication unit configured to communicate with an external device, and the processor may be configured to control the communication unit to transmit the generated image to the external device and to display the image.

According to an embodiment of the present disclosure, a control method of a probe device including: a matrix array AFE including a plurality of cells, and outputting an electric signal corresponding to each of the plurality of cells, and a transducer unit to transduce the electric signal outputted from each of the plurality of cells into an ultrasound signal, may include: grouping the plurality of cells into at least one group corresponding to at least one diagnosis mode; and controlling cells corresponding to each group to output an ultrasound signal having a different characteristic according to a corresponding diagnosis mode through the transducer unit.

The control method of the probe device according to an embodiment of the present disclosure may further include transmitting the ultrasound signal having the different characteristic to a target, and generating a different image based on an ultrasound signal received from the target, or transmitting a focused ultrasound signal to the target.

In addition, the controlling may include controlling cells corresponding to each diagnosis mode to adjust at least one of a size, a frequency, and a focusing point of the ultrasound signal, and to output a different ultrasound signal.

Herein, the diagnosis mode may include a first mode for generating a second harmonic image, a second mode for generating a plurality of images related to a plurality of targets, a third mode for transmitting a focused ultrasound signal, and a fourth mode for transmitting a high-voltage/low-voltage ultrasound signal.

The control method of the probe device according to an embodiment of the present disclosure may further include, in the first mode, dividing a plurality of cells belonging to a group corresponding to the first mode into a first transmission and reception region and a second transmission and reception region, transmitting a first ultrasound signal and a second ultrasound signal having a phase difference of 180 degrees to a target, simultaneously, through the first transmission and reception region and the second transmission and reception region, and generating the second harmonic image based on the first ultrasound signal and the second ultrasound signal received from the target.

In addition, the control method of the probe device according to an embodiment of the present disclosure may further include, in the second mode, dividing a plurality of cells belonging to a group corresponding to the second mode into a plurality of transmission and reception regions to transmit and receive the ultrasound signal to and from the plurality of targets, and detecting an abnormal portion by comparing images generated from the plurality of transmission and reception regions.

In addition, the control method of the probe device according to an embodiment of the present disclosure may further include, in the third mode, dividing a plurality of cells belonging to a group corresponding to the third group into a first transmission region and a second transmission and reception region, transmitting a focused ultrasound signal for a treatment to a target through the first transmission region, transmitting and receiving the ultrasound signal to and from the target through the second transmission and reception region, and generating an image regarding a progress of a treatment by the focused ultrasound signal.

In addition, the control method of the probe device according to an embodiment of the present disclosure may further include, in the fourth mode, dividing a plurality of cells belonging to a group corresponding to the fourth mode into a first transmission and reception region for generating and transmitting and receiving a high-voltage ultrasound signal, and a second transmission and reception region for generating and transmitting and receiving a low-voltage ultrasound signal, generating a first image based on the transmitted and received high-voltage ultrasound signal, and generating a second image based on the transmitted and received low-voltage ultrasound signal.

In addition, the control method of the probe device according to an embodiment of the present disclosure may further include performing beamforming with respect to the plurality of cells belonging to each group, based on at least one of a depth, a size, and a location of a target, such that an ultrasound signal outputted through the transducer unit is focused onto the target.

### Advantageous Effect

According to various embodiments of the present disclosure as described above, various functions can be supported by using one probe device.

### BRIEF DESCRIPTION OF THE DRAWINGS

FIG. 1 is a view to illustrate a related-art technology;

FIG. 2 is a block diagram showing a configuration of a probe device according to an embodiment of the present disclosure;

FIG. 3 is a view showing a detailed configuration of a matrix array AFE according to an embodiment of the present disclosure;

FIG. 4 is a view showing a schematic configuration of a probe device according to an embodiment of the present disclosure;

FIGS. 5 to 7B are views to illustrate a process of generating a second harmonic image according to an embodiment of the present disclosure;

FIG. 8 is a view related to a probe device using a 1D transducer according to an embodiment of the present disclosure;

FIGS. 9 to 11 are views to illustrate a process of generating a plurality of images regarding a plurality of targets according to an embodiment of the present disclosure;

FIGS. 12 to 14 are views to illustrate a process of transmitting a focused ultrasound signal according to an embodiment of the present disclosure;

FIGS. 15 to 17 are views to illustrate a process of transmitting a high-voltage/low-voltage ultrasound signal according to an embodiment of the present disclosure;

FIG. 18 is a block diagram showing a configuration of a probe device according to another embodiment of the present disclosure;

FIG. 19 is a block diagram showing a configuration of a probe device according to still another embodiment of the present disclosure;

FIG. 20 is a block diagram showing a detailed configuration of the probe device 100 shown in FIG. 2;

FIG. 21 is a view to illustrate a software module stored in a storage according to an embodiment of the present disclosure;

FIG. 22 is a flowchart to illustrate a control method of a probe device according to an embodiment of the present disclosure; and

FIGS. 23 to 25 are views to illustrate images generated according to respective modes according to an embodiment of the present disclosure.

### BEST MODE FOR EMBODYING THE INVENTION

Hereinafter, the present disclosure will be described in great detail with reference to the accompanying drawings. In the following description, detailed descriptions of well-known functions or configurations will be omitted since they would unnecessarily obscure the subject matters of the present disclosure. Also, the terms used herein are defined according to the functions in the present disclosure. Thus, the terms may vary depending on user's or operator's intension and usage. That is, the terms used herein must be understood based on the descriptions made herein.

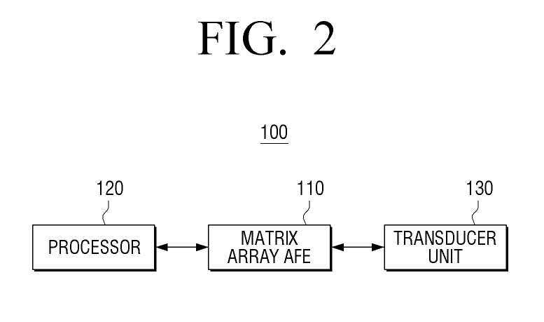

FIG. 2 is a block diagram showing a configuration of a probe device according to an embodiment of the present disclosure.

Referring to FIG. 2, the probe device 100 includes a matrix array analog front end (AFE) 110, a processor 120, and a transducer unit 130. Herein, the probe device 100 is generally connected to a main body of an ultrasound diagnosis device and is brought into contact with an examination portion of an examinee, and performs a role of transmitting and receiving ultrasound signals to and from the examinee. However, according to an embodiment of the present disclosure, the probe device 100 may perform only the role of transmitting and receiving ultrasound signals to and from the examinee, or may perform not only the role of transmitting and receiving ultrasound signals, but also a role of generating an image based on the received ultrasound signal. That is, a related-art ultrasound examination system is divided into a main body of an ultrasound diagnosis device and a probe, whereas the probe device 100 according to an embodiment of the present disclosure may include only a probe or may include both the main body of the related-art ultrasound diagnosis device and the probe.

The matrix array AFE 110 may include a plurality of cells and may output an electric signal corresponding to each of the plurality of cells. Specifically, the matrix array AFE 110 may include a plurality of cells arranged in the form of a matrix array, and each of the plurality of cells may include a delay circuit which is able to transmit and receive an electric signal, and is related to beamforming, and a circuit for amplifying and filtering.

That is, the matrix array AFE 110 may be defined as an analog front end circuit in the form of a matrix array, which includes a plurality of cells arranged in the form of a matrix array, and includes various circuits for each of the plurality of cells.

The matrix array AFE 110 may be used for the probe device 100 because the plurality of cells included in the matrix array AFE 110 may be divided at user's discretion and each of the divided cells may be controlled to perform a different function.

The transducer unit 130 may transduce, into an ultrasound signal, an electric signal outputted from each of the plurality of cells included in the matrix array AFE 110. Herein, the transducer unit 130 may refer to a conversion device that converts an input signal into an output signal of a different form, and in particular, may refer to a transducer that transduces alternating current energy of hundreds of Hz or more into a mechanical vibration of the same frequency. Accordingly, the transducer unit 130 may transduce the electric signal outputted from each of the plurality of cells included in the matrix array AFE 110 into the ultrasound signal corresponding to each of the plurality of cells, and may output the transduced signal.

The processor 120 may group the plurality of cells into at least one group corresponding to at least one diagnosis mode, and may control such that cells corresponding to each group output, through the transducer unit 130, an ultrasound signal having a different characteristic according to a corresponding diagnosis mode.

For example, the processor 120 may group the plurality of cells included in the matrix array AFE 110 into two groups, and in this case, the groups may operate in different diagnosis modes. Accordingly, the processor 120 may control a first group of the two groups to operate in a first diagnosis mode, and accordingly, the first group may cause an ultrasound signal having a characteristic to be used in the first diagnosis mode to be outputted through the transducer unit 130. In addition, the processor 120 may control a second group of the two groups to operate in a second diagnosis mode, and accordingly, the second group may cause an ultrasound signal having a characteristic to be used in the second diagnosis mode to be outputted through the transducer unit 130.

Although the processor 120 groups the plurality of cells included in the matrix array AFE 110 into two groups in the above-described example, the processor 120 may group the plurality of cells into three or four groups, and the number of groups may be changed according to the number of selected diagnosis modes.

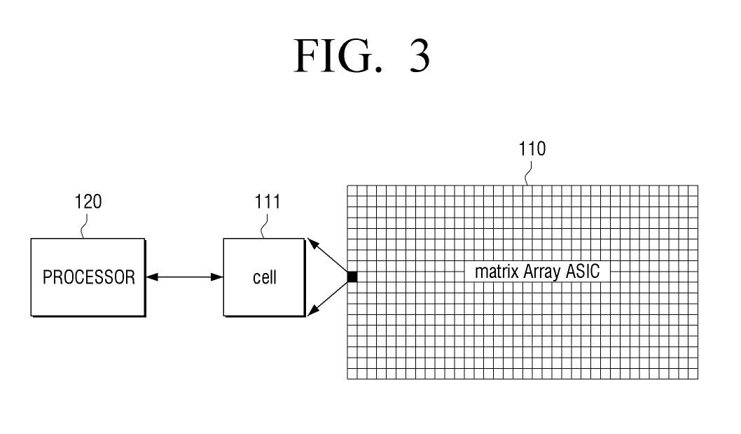

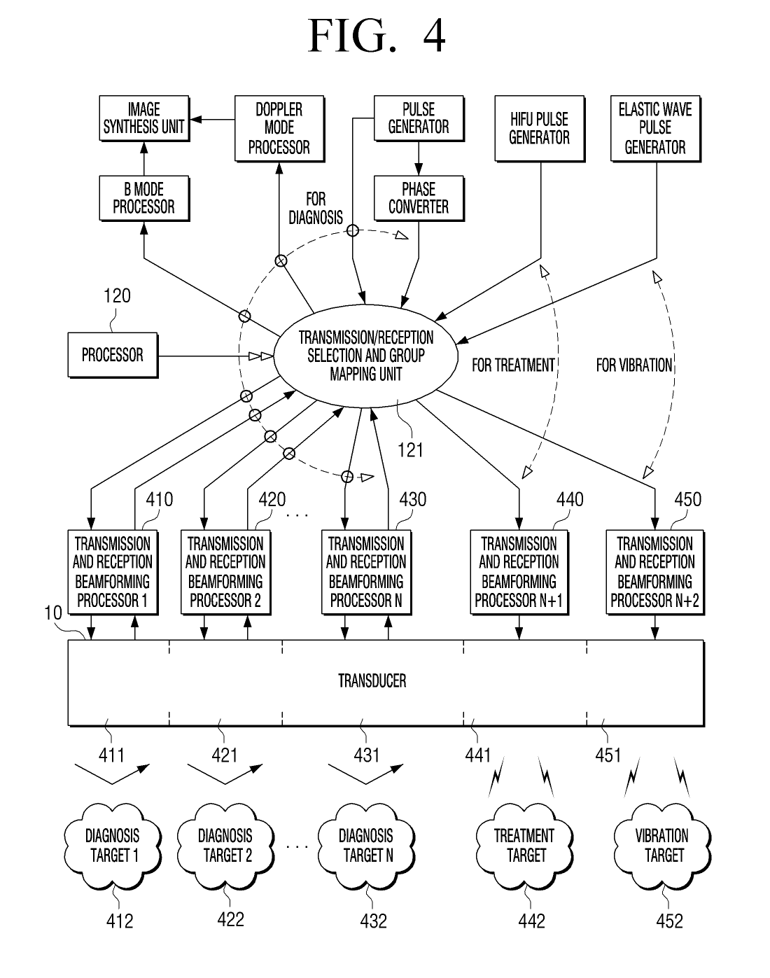

The plurality of cells included in the matrix array AFE 110 will be described in detail with reference to FIG. 3.

FIG. 3 is a view showing a detailed configuration of the matrix array AFE according to an embodiment of the present disclosure.

Referring to FIG. 3, the matrix array AFE 110 may include the plurality of cells arranged in the form of a matrix array. Herein, the plurality of cells may be implemented by using different aperture regions, and each of the plurality of cells may output an electric signal to be transduced into an ultrasound signal. Specifically, the cell is a unit element for forming the matrix array AFC 110, and an electric signal outputted from the cell may be transmitted to the transducer unit 130, and the transducer unit 130 may transduce the electric signal transmitted from the cell into an ultrasound signal.

The processor 120 may individually control each of the plurality of cells forming the matrix array AFE 110, and it can be seen from FIG. 3 that the processor 120 controls a cell 111. The one cell 111 may output an electric signal to be transduced into an ultrasound signal at the transducer unit 130, and the processor 120 may adjust data included in the electric signal outputted from the cell 111, and may control such that the electric signal having the adjusted data is transduced into an ultrasound signal having a characteristic corresponding to each diagnosis mode and the ultrasound signal is outputted.

Specifically, in adjusting data included in the electric signal outputted from the cell 111, the processor 120 may adjust at least one of, for example, data for turning on/off the cell 111, data for selecting a diagnosis mode, data related to beamforming, and apodization data. Herein, the data related to the beamforming refers to data that causes the ultrasound signal outputted through the transducer unit 130 to be focused onto a target point. In addition, the apodization data refers to data related to processing for reducing a high-order diffraction figure.

Accordingly, the processor 120 may adjust various data related to the cell 111 and group the plurality of cells into one group, and may reconfigure the plurality of cells such that each group performs a function corresponding to a different diagnosis mode.

In FIG. 3, the matrix array AFE 110 is indicated by the matrix array ASIC. Herein, the ASIC (application specific integrated circuit) refers to a customized integrated circuit for using for a specific purpose, and the matrix array ASIC used in the drawing is defined as having the same meaning as the matrix array AFE.

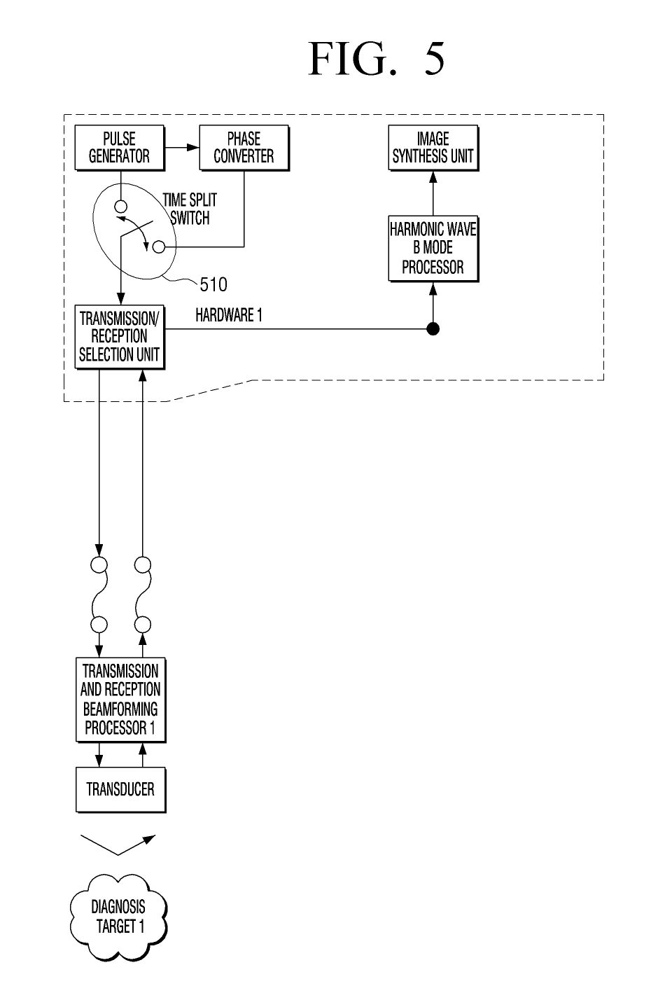

FIG. 4 is a view showing a schematic configuration of a probe device according to an embodiment of the present disclosure.

Referring to FIG. 4, the probe device 100 includes a plurality of transmission and reception beamforming processors 410, 420, 430, 440, 450, and a transducer 10. Herein, each region of the transducer 10 may correspond to each of the plurality of transmission and reception beamforming processors. For example, the transmission and reception beamforming processor 1 410 may correspond to a first region 411 of the transducer 10, the transmission and reception beamforming processor 2 420 may correspond to a second region 421 of the transducer 10, the transmission and reception beamforming processor N 430 may correspond to a N-th region 431 of the transducer 10, the transmission and reception beamforming processor N+1 440 may correspond to a N+1-th region 441 of the transducer 10, and the transmission and reception beamforming processor N+2 450 may correspond to a N+2-th region 451 of the transducer 10.

In addition, the transmission and reception beamforming processor 1 410, the transmission and reception beamforming processor 2 420, the transmission and reception beamforming processor N 430, the transmission and reception beamforming processor N+1 440, and the transmission and reception beamforming processor N+2 450 may correspond to groups into which a plurality of cells included in the matrix array AFE 110 are grouped.

In addition, an ultrasound signal outputted from the first region 411 of the transducer 10 may be focused onto a diagnosis target 1 412, an ultrasound signal outputted from the second region 421 of the transducer 10 may be focused onto a diagnosis target 2 422, an ultrasound signal outputted from the N-th region 431 of the transducer 10 may be focused onto a diagnosis target N 432, and an ultrasound signal outputted from the N+1-th region 441 of the transducer 10 may be focused onto a treatment target 442.

As described above, the probe device 100 may focus and output the ultrasound signals to the plurality of targets, or may focus and output the ultrasound signals not only to the plurality of targets, but also to different points in one target. That is, different points of one target onto which the ultrasound signals are focused are defined as focusing points, and the operation of the probe device 100 described in the present disclosure is equally applied to the plurality of focusing points existing in one target. For example, the ultrasound signal outputted from the first region 411 of the transducer 10 may be focused onto a first focusing point of a target, the ultrasound signal outputted from the second region 421 of the transducer 10 may be focused onto a second focusing point of the target, and the ultrasound signal outputted from the N-th region 431 of the transducer 10 may be focused onto an N-th focusing point of the target.

In addition, the processor 120 may control a transmission/reception selection and group mapping unit 121 to group the plurality of cells forming the matrix array AFE 110 into at least one group corresponding to at least one diagnosis mode, and to select a transmission and reception beamforming processor corresponding to each group, and may control such that each region of the transducer 10 outputs an ultrasound signal corresponding to each diagnosis mode to a corresponding diagnosis target.

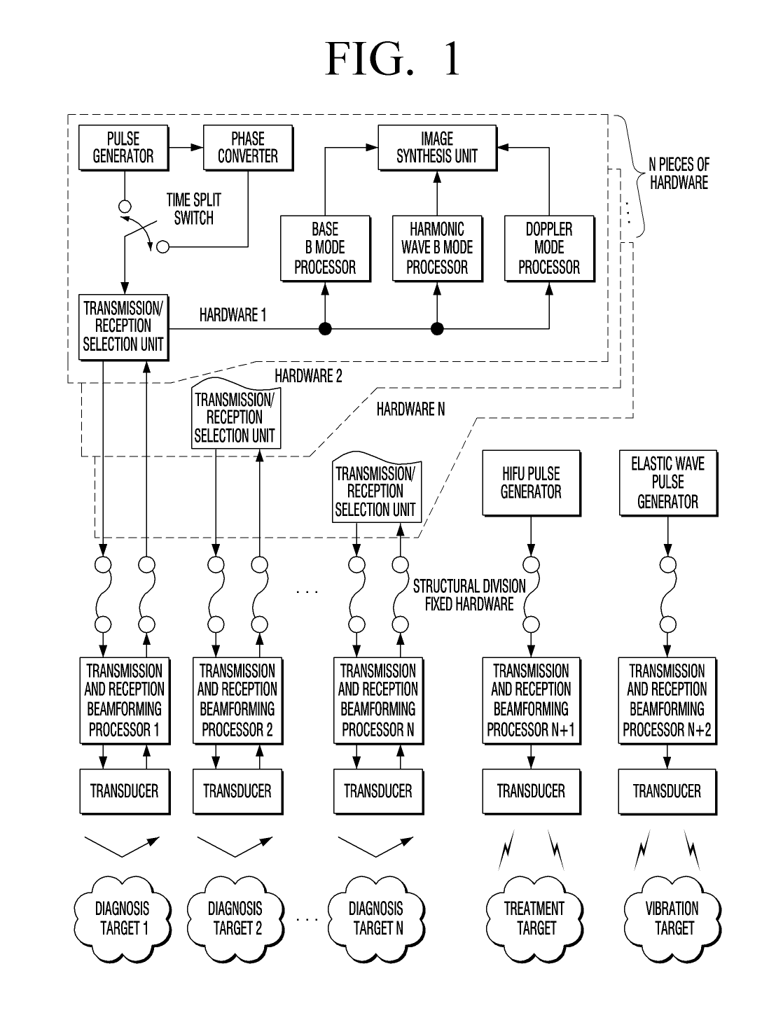

As described above, the probe device 100 shown in FIG. 4 may group the plurality of cells forming the matrix array AFE, provided in one probe device, into the plurality of groups, and may control such that the groups perform functions corresponding to different diagnosis modes simultaneously. In terms of this, there is an effect of reducing inconvenience of having to change to another probe device to perform a different function as in the related-art ultrasound examination system shown in FIG. 1.

The processor 120 may transmit an ultrasound signal having a different characteristic to a target, and may generate a different image based on an ultrasound signal received from the target, or may transmit a focused ultrasound signal to the target.

For example, the processor 120 may transmit an ultrasound signal for a diagnosis to a target, and may generate an image based on an ultrasound signal received from the target, or may transmit an ultrasound wave for a treatment to a target and may perform a treatment function.

In addition, in transmitting an ultrasound signal for a diagnosis to a target, the processor 120 may transmit ultrasound signals having characteristics corresponding to various types of diagnosis modes to targets.

Specifically, the processor 120 may control to adjust at least one of a size, a frequency, and a focusing point of an ultrasound signal in a cell corresponding to each diagnosis mode, and to output the ultrasound signal.

Since a size, a frequency, and a focusing point of a necessary ultrasound signal vary according to each diagnosis mode, the processor 120 may control to adjust at least one of the size, the frequency, and the focusing point of the ultrasound signal according to a diagnosis mode, and to output an ultrasound signal having a characteristic corresponding to the diagnosis mode.

In addition, the processor 120 may group the plurality of cells forming the matrix array AFE 110 to suit each diagnosis mode according to a depth, a size, and a location of a target. In addition, the processor 120 may control the groups to perform different functions.

The diagnosis mode may include a first mode for generating a second harmonic image, a second mode for generating a plurality of images related to a plurality of targets, a third mode for transmitting a focused ultrasound signal, and a fourth mode for transmitting a high-voltage/low-voltage ultrasound signal. Of course, the diagnosis mode is not limited to the above-described four modes, and may include other various diagnosis or treatment modes.

A process of operating the probe device 100 according to each diagnosis mode will be described in detail.

[First Mode for Generating a Second Harmonic Image]

FIGS. 5 to 7 are views to illustrate a process of generating a second harmonic image according to an embodiment of the present disclosure.

In particular, FIG. 5 illustrates a related-art technology used for generating a second harmonic image. Referring to FIG. 5, a related-art ultrasound examination system normally generates a multi-line transmission technique to generate a second harmonic image. The multi-line transmission technique is to generate a second harmonic image by transmitting and receiving two pulses and synthesizing two received pulses. However, since one probe device of the related-art ultrasound examination system includes only one transmission and reception beamforming processor, the probe device is not able to transmit two pulses to a target simultaneously, and has no choice but to transmit a first pulse to the target first, and after an interval, to transmit a second pulse to the target. Likewise, there may be a time lag between reception of the first pulse and reception of the second pulse from the target. Accordingly, time is required to generate the second harmonic image, and a frame rate of an image per the same time may be reduced. In FIG. 5, a time split switch 510 is required to transmit the first pulse and the second pulse to the target at intervals.

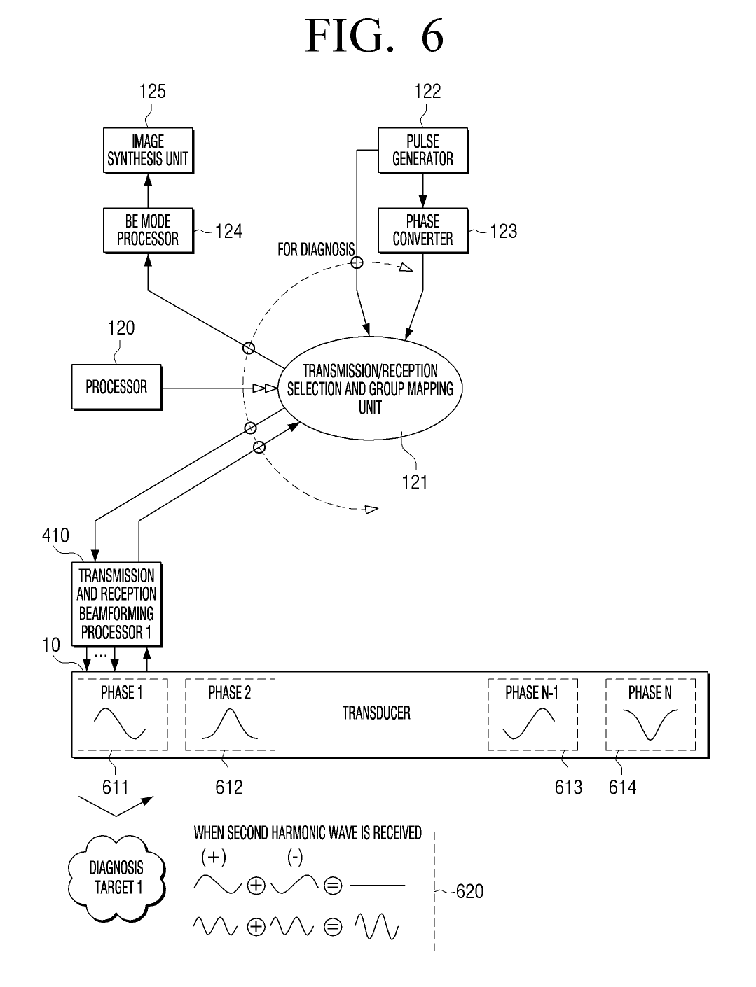

FIG. 6 illustrates a process of generating a second harmonic image according to an embodiment of the present disclosure. Referring to FIG. 6, the processor 120 may control the transmission and reception beamforming processor 1 410 through the transmission/reception selection and group mapping unit 121 to transmit pulses having different phases simultaneously.

In particular, in the first mode for generating the second harmonic image, the processor 120 may divide a plurality of cells belonging to a group corresponding to the first mode into a first transmission and reception region and a second transmission and reception region, and may transmit a first ultrasound signal and a second ultrasound signal having a phase difference of 180 degrees to a target, simultaneously, through the first transmission and reception region and the second transmission and reception region, and may generate the second harmonic image based on the first ultrasound signal and the second ultrasound signal received from the target.

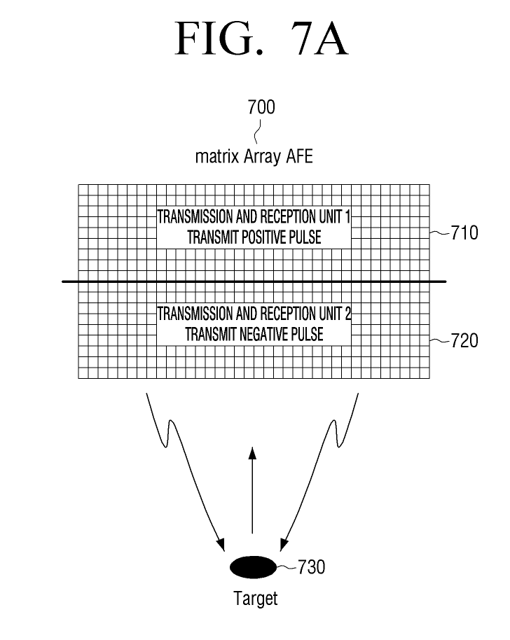

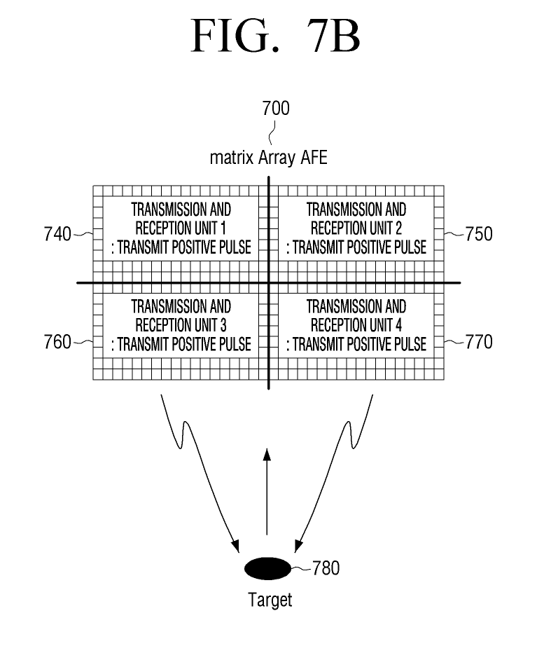

Specifically, referring to FIG. 7A, a plurality of cells included in a matrix array AFE 700 may be divided into a transmission and reception unit 1 710 and a transmission and reception unit 2 720. In addition, the processor 120 may transmit two pulses having a phase difference of 180 degrees to a target 730, simultaneously, through the transmission and reception unit 1 710 and the transmission and reception unit 2 720, and may receive two pulses from the target 730 simultaneously.

That is, the processor 120 may group the plurality of cells included in the matrix array AFE 700 into two groups 710, 720 to generate a second harmonic image, and cells corresponding to the groups may transmit and receive pulses having a phase difference of 180 degrees to the target.

In addition, the processor 120 may offset fundamental frequency (fo) components by synthesizing the two pulses having the phase difference of 180 degrees, received from the target 730, and may detect only a second harmonic (2*fo) component.

Accordingly, the processor 120 may generate a second harmonic image based on the detected second harmonic (2*fo) component.

Referring to FIG. 7B, the plurality of cells included in the matrix array AFE 700 may be divided into a transmission and reception unit 1 740, a transmission and reception unit 2 750, a transmission and reception unit 3 760, and a transmission and reception unit 4 770. In addition, the processor 120 may transmit two pulses having a phase difference of 180 degrees to a target 780, simultaneously, through the transmission and reception unit 1 740, the transmission and reception unit 2 750, the transmission and reception unit 3 760, and the transmission and reception unit 4 770, and may receive two pulses from the target 780 simultaneously.

For example, the processor 120 may control the transmission and reception unit 1 740 and the transmission and reception unit 4 770 to transmit pulses having a phase of 0 degree to the target 780 simultaneously, and to receive the same pulses from the target 780, and may control the transmission and reception unit 2 750 and the transmission and reception unit 3 760 to transmit pulses having a phase of 180 degrees to the target 780 simultaneously, and to receive the same pulses from the target 780.

That is, the processor 120 may group the plurality of cells included in the matrix array AFE 700 into four groups 740, 750, 760, 770 to generate a second harmonic image, and cells corresponding to the groups may transmit and receive the pulse having the phase of 0 degree and the pulse having the phase of 180 degrees to and from the target 780.

In addition, the processor 120 may offset fundamental frequency (fo) components by synthesizing the pulse having the phase of 0 degree and the pulse having the phase of 180 degrees, received from the target 780, and may detect only a second harmonic (2*fo) component.

Accordingly, the processor 120 may generate a second harmonic image based on the detected second harmonic (2*fo) component.

Referring back to FIG. 6, the processor 120 may control the transmission/reception selection and group mapping unit 121 to group the plurality of cells included in the matrix array AFE 110 into, for example, a first group and a second group, and may control the first group and the second group through the transmission and reception beamforming processor 1 410.

Herein, the first group and the second group may correspond to a first region and a second region forming the transducer 10, and the first region may transmit an ultrasound signal 611 having a phase 1 to a diagnosis target 1, and the second region may transmit an ultrasound signal 613 of a phase N−1 having a phase difference of 180 degrees from the phase 1 to the diagnosis target 1.

In addition, in response to ultrasound signals being received from the diagnosis target 1, fundamental frequency (fo) components may be offset by each other due to the phase difference as indicated by a box portion 620 expressed by a dashed line, and only a second harmonic (2*fo) component may be extracted.

In addition, the processor 120 may transmit the detected second harmonic (2*fo) component to a B mode processor 124, and the B mode processor 124 may generate an image by processing the second harmonic (2*fo) component, and an image synthesis unit 125 may generate one synthesis image by synthesizing the generated images.

The ultrasound signal 611 having the phase 1 and the ultrasound signal 613 having the phase N−1 may be generated by generating pulses from a pulse generator 122, and converting phases of the generated pulses to have a phase difference of 180 degrees through a phase converter 123.

Likewise, the first region may transmit an ultrasound signal 612 having a phase 2 to the diagnosis target 1, and the second region may transmit an ultrasound signal 614 of a phase N having a phase difference of 180 degrees from the phase 2 to the diagnosis target 1.

In addition, in response to ultrasound signals being received from the diagnosis target 1, fundamental frequency (fo) components may be offset by each other due to the phase difference as indicated by the box portion 620 expressed by the dashed line, and only a second harmonic (2*fo) component may be extracted.

In addition, the processor 120 may transmit the detected second harmonic (2*fo) component to the B mode processor 124, and the B mode processor 124 may generate an image by processing the second harmonic (2*fo) component, and the image synthesis unit 125 may generate one synthesis image by synthesizing the generated images.

As described above, by dividing the plurality of cells included in the matrix array AFE 110 into the plurality of groups, transmitting pulses having the phase difference of 180 degree to a target simultaneously, and generating a second harmonic image by detecting a second harmonic component based on the pulses received from the target, the process of the present disclosure can increase a frame rate two times in comparison to the related-art method that transmits and receives a pulse of 0 degree and then transmits and receives a pulse of 180 degrees, and synthesizes two images. In addition, as the frame rate increases two times and a processing speed increases, a motion artifact regarding a motion of the target may decelerate. In addition, a band-pass filter for separating the second harmonic image may be removed and a cut-off characteristic may be alleviated. Therefore, a design scheme can be simplified.

The above-described process of obtaining the second harmonic image may be equally applied to a related-art probe device using a 1D transducer. That is, on the assumption that there is a scan line in the middle of apertures, the 1D transducer may be divided in the vertical direction, that is, may be divided into a first group on the left and a second group on the right, and the first group transmits a pulse having a phase of 0 degree to a target, and the second group transmits a pulse having a phase of 180 degrees to the target, and a second harmonic image may be generated based on the pulses received from the target.

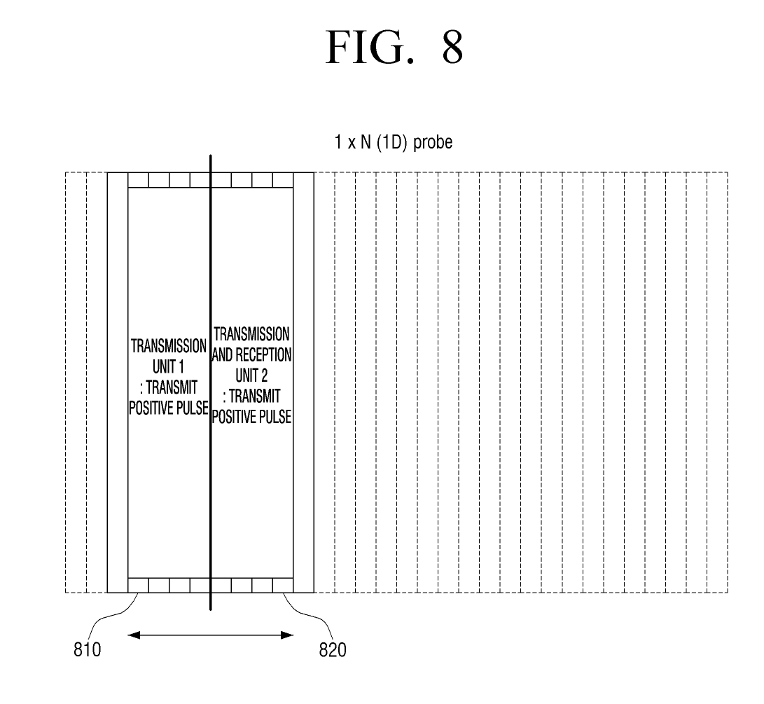

FIG. 8 is a view related to a probe device using a 1D transducer according to an embodiment of the present disclosure.

Referring to FIG. 8, a scan line may be configured, while sliding on an aperture surface of the 1D transducer, and the aperture surface may be divided into a transmission and reception unit 1 810 and a transmission and reception unit 2 820. The transmission and reception unit 1 810 and the transmission and reception unit 2 820 may transmit a first pulse and a second pulse having a phase difference of 180 degrees to a target simultaneously, and receive pulses from the target, such that the processor 120 can generate a second harmonic image based on the received first and second pulses.

The first mode for generating the second harmonic image may be implemented by a B-mode, and the B-mode is well known technology and thus a detailed description thereof is omitted.

Although the description of FIGS. 5 to 7B is related to generation of the second-harmonic image, the description may be applied to a method for obtaining third or higher order harmonic images by changing a phase combination of ultrasound signals. For example, when four different phases of the ultrasound signals 611-614 in FIG. 6 are combined, a fourth harmonic image may be generated.

[Second Mode for Generating a Plurality of Images Related to a Plurality of Targets]

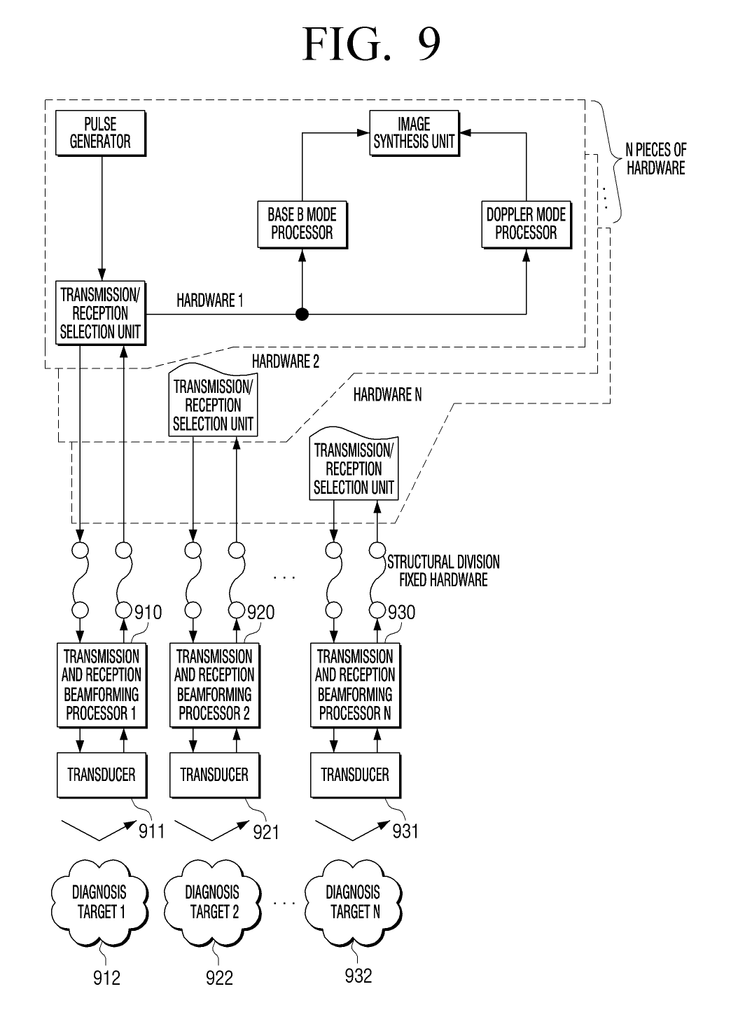

FIGS. 9 to 11 are views to illustrate a process of generating a plurality of images related to a plurality of targets according to an embodiment of the present disclosure.

In particular, FIG. 9 illustrates a related-art technology used for generating a plurality of images related to a plurality of targets. Referring to FIG. 9, a related-art ultrasound examination system requires a plurality of probe devices according to the number of diagnosis targets. As shown in FIG. 9, all probe devices corresponding to diagnosis targets 1, 2, 3 912, 922, 932 are required.

For example, a first probe device including a first transducer 911, a transmission and reception beamforming processor 1 910, a pulse generator, a transmission/reception selection unit, a base B mode processor, an image synthesis unit, a Doppler mode processor, etc. may be required to transmit and receive an ultrasound signal to and from the diagnosis target 1 912, a second probe device including a second transducer 921, a transmission and reception beamforming processor 2 920, a pulse generator, a transmission/reception selection unit, a base B mode processor, an image synthesis unit, a Doppler mode processor, etc. may be required to transmit and receive an ultrasound signal to and from the diagnosis target 2 922, and an N-th probe device including an N-th transducer 931, a transmission and reception beamforming processor N 930, a pulse generator, a transmission/reception selection unit, a base B mode processor, an image synthesis unit, a Doppler mode processor, etc. may be required to transmit and receive an ultrasound signal to the diagnosis target N 932.

Accordingly, when the related-art ultrasound examination system has the plurality of diagnosis targets to be examined simultaneously, there is no choice but to require the plurality of probe devices.

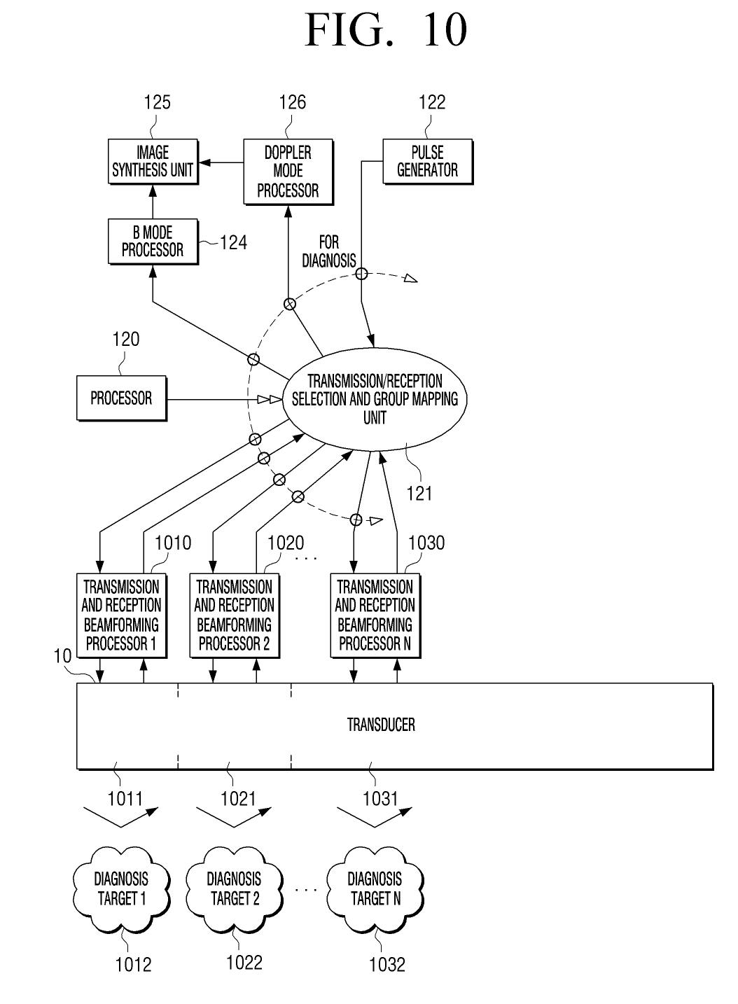

FIG. 10 illustrates a process of generating a plurality of images related to a plurality of targets according to an embodiment of the present disclosure. Referring to FIG. 10, the processor 120 may control a transmission and reception beamforming processor 1 1010, a transmission and reception beamforming processor 2 1020, and a transmission and reception beamforming processor N 1030 through a transmission/reception selection and group mapping unit 121 to transmit ultrasound signals to different diagnosis targets 1, 2, N 1012, 1022, 1032 simultaneously.

In particular, in the second mode for generating the plurality of images related to the plurality of targets, the processor 120 may divide a plurality of cells belonging to a group corresponding to the second mode into a plurality of transmission and reception regions to transmit and receive ultrasound signals to and from the plurality of targets, and may detect an abnormal portion by comparing images generated from the plurality of transmission and reception regions.

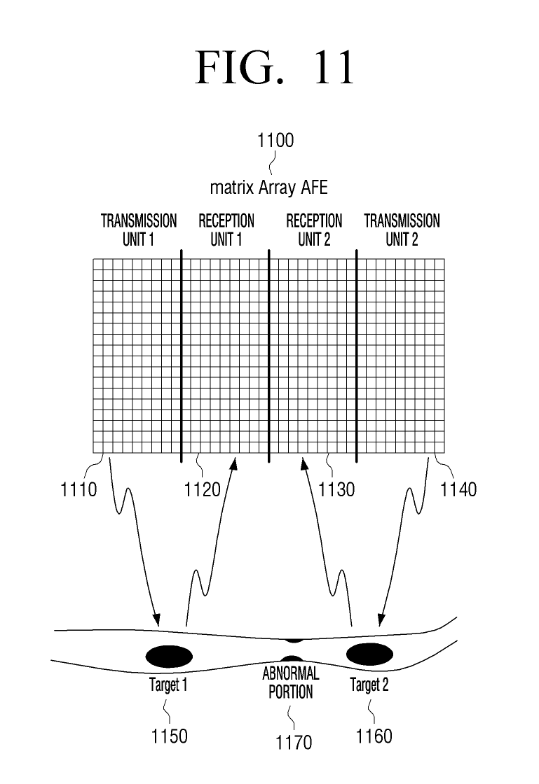

Specifically, referring to FIG. 11, when it is assumed that all of the plurality of cells included in a matrix array AFE 1100 are grouped into a group corresponding to the second mode for generating a plurality of images related to a plurality of targets, it can be known that the plurality of cells belonging to the group corresponding to the second mode are divided into a transmission unit 1 1110, a reception unit 1 1120, a reception unit 2 1130, and a transmission unit 2 1140.

In addition, the processor 120 may transmit a first ultrasound signal to a first target 1150 through the transmission unit 1 1110, and may receive the first ultrasound signal from the first target 1150 through the reception unit 1 1120.

In addition, the processor 120 may transmit a second ultrasound signal to a second target 1160 through the transmission unit 2 1140, and may receive the second ultrasound signal from the second target 1160 through the reception unit 2 1130.

In addition, the processor 120 may generate a first image regarding the first target 1150 and a second image regarding the second target 1160, based on the first ultrasound signal received from the first target 1150 and the second ultrasound signal received from the second target 1160, respectively.

In addition, the processor 120 may detect an abnormal portion 1170 by comparing the first image and the second image. For example, the processor 120 may compare a first blood flow rate detected through the first image and a second blood flow rate detected through the second image, and, in response to a first blood flow rate pattern and a second blood flow rate pattern being different from each other as a result of comparing, it may be determined that there is an angiostenosis between the first target 1150 and the second target 1160, or there is a foreign substance.

Referring back to FIG. 10, the processor 120 may control the transmission/reception selection and group mapping unit 121 to group the plurality of cells included in the matrix array AFE 110 into a first group, a second group, and a third group corresponding to a diagnosis target 1 1012, a diagnosis target 2 1022, and a diagnosis target N 1032.

Herein, the first group may correspond to a transmission and reception beamforming processor 1 1010 and a first region 1011 of the transducer 10, the second group may correspond to a transmission and reception beamforming processor 2 1020 and a second region 1021 of the transducer 10, and the third group may correspond to a transmission and reception beamforming processor N 1030 and an N-th region 1031 of the transducer 10.

In addition, the first region 1011 of the transducer 10 may transmit and receive an ultrasound signal to and from the diagnosis target 1 1012, the second region 1021 of the transducer 10 may transmit and a receive an ultrasound signal to and from the diagnosis target 2 1022, and the N-th region 1031 of the transducer 10 may transmit and receive an ultrasound signal to and from the diagnosis target N 1032.

In addition, in response to the ultrasound signals being received from the diagnosis target 1 1012, the diagnosis target 2 1022, and the diagnosis target N 1032, the respective ultrasound signals may be transmitted to the B mode processor 124 through the transmission and reception beamforming processor 1 1010, the transmission and reception beamforming processor 2 1020, and the transmission and reception beamforming processor N 1030, and the B mode processor 124 may generate a first image, a second image, and a third image based on the received ultrasound signals, and a Doppler mode processor 126 and the image synthesis unit 125 may determine an abnormal portion by comparing and analyzing the first image, the second image, and the third image, and may generate a synthesis image to predict the abnormal portion.

The pulse generator 122 may generate pulses to be used to output the ultrasound signals at the respective regions 1011, 1021, 1031 of the transducer 10.

As described above, the probe device 100 according to an embodiment of the present disclosure may determine an abnormal portion by transmitting and receiving ultrasound signals to and from ae plurality of diagnosis targets simultaneously, and generating respective images, and may reduce inconvenience of a related-art ultrasound examination system that requires a plurality of probe devices to examine a plurality of diagnosis targets simultaneously.

[Third Mode for Transmitting a Focused Ultrasound Signal]

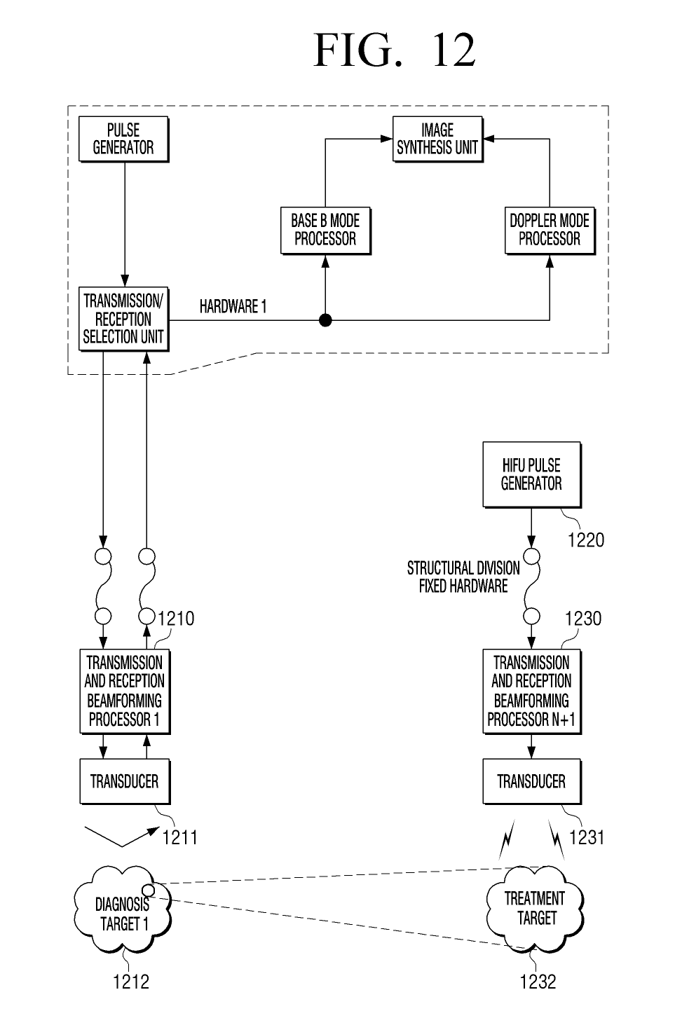

FIGS. 12 to 14 are views to illustrate a process of transmitting a focused ultrasound signal according to an embodiment of the present disclosure. In particular, FIG. 12 illustrates a related-art technology used for transmitting a focused ultrasound signal. Referring to FIG. 12, a related-art ultrasound examination system may separately include a probe device for a diagnosis target, and a treatment device for a treatment target.

For example, the related-art ultrasound examination system separately requires a probe device including a transducer 1211 for transmitting and receiving ultrasound signals to and from a diagnosis target 1 1212, a transmission and reception beamforming processor 1 1210, a pulse generator, a transmission/reception selection unit, a base B mode processor, an image synthesis unit, a Doppler mode processor, etc., and a treatment device including a transducer 1231 for transmitting and receiving focused ultrasound signals to and from a treatment target 1232, a transmission and reception beamforming processor N+1 1230, and a high intensity focused ultrasound (HIFU) pulse generator 1220.

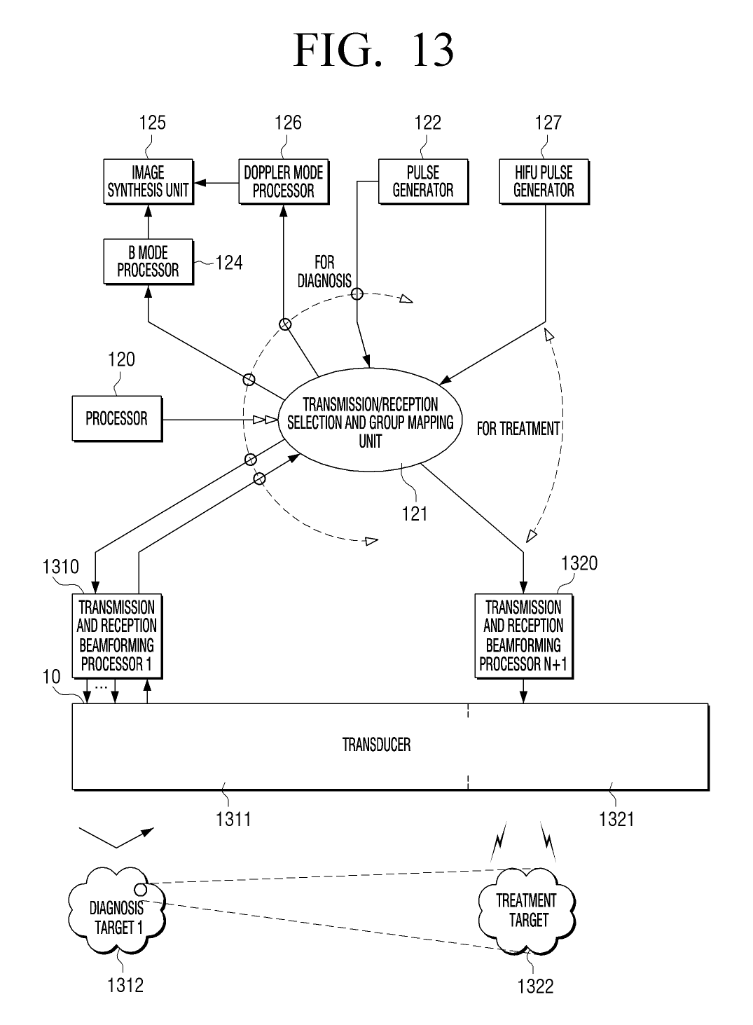

FIG. 13 illustrates a process of transmitting a focused ultrasound signal according to an embodiment of the present disclosure. Referring to FIG. 13, the processor 120 may control a transmission and reception beamforming processor 1 1310 through the transmission/reception selection and group mapping unit 121 to transmit an ultrasound signal to a diagnosis target 1 1312, and may control an HIFU pulse generator 127 and a transmission and reception beamforming processor N+1 1320 to transmit a focused ultrasound signal to a treatment target 1322.

In particular, in the third mode for transmitting the focused ultrasound signal, the processor 120 may divide a plurality of cells belonging to a group corresponding to the third mode into a first transmission region and a second transmission and reception region, and may transmit a focused ultrasound signal for a treatment to a target through the first transmission region, and may transmit and receive ultrasound signals to and from the target through the second transmission and reception region and may generate an image related to a progress of a treatment by the focused ultrasound signal.

Herein, the processor 120 may freely change the first transmission region and the second transmission region according to a depth or a size of the target in order to achieve an optimal effect and obtain an exact image.

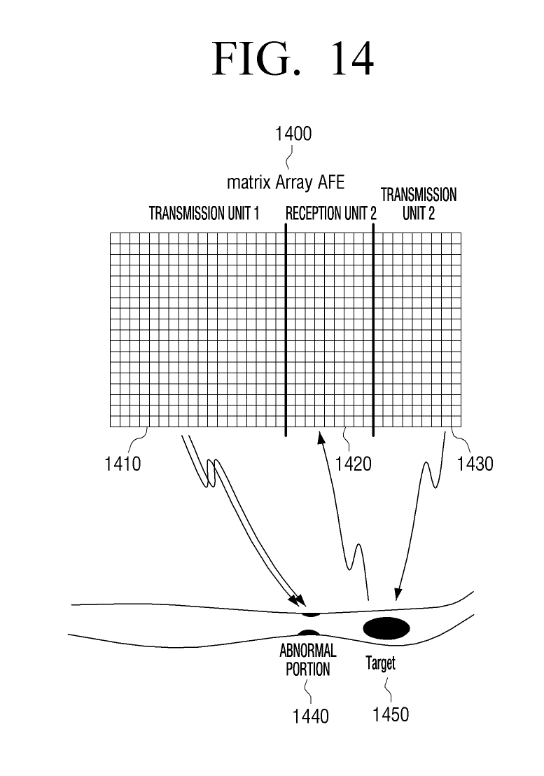

Specifically, referring to FIG. 14, when it is assumed that all of the plurality of cells included in a matrix array AFE 1400 are grouped into a group corresponding to the third mode for transmitting a focused ultrasound signal, it can be known that the plurality of cells belonging to the group corresponding to the third mode are divided into a transmission unit 1 1410, a reception unit 2 1420, and a transmission unit 2 1430.

In addition, the processor 120 may transmit a focused ultrasound signal to an abnormal portion 1440, which is a target, through the transmission unit 1 1410, and may transmit an ultrasound signal to a target 1450 through the transmission unit 2 1430, and may receive an ultrasound signal from the target 1450 through the reception unit 2 1420.

Herein, the focused ultrasound signal may be implemented by an HIFU, and the focused ultrasound signal may cut off or destroy the abnormal portion or may perform a disinfection function, and thus may be used for a treatment.

In addition, the processor 120 may generate an image regarding the target 1450 based on the ultrasound signal received from the target 1450.

Accordingly, the processor 120 may determine the progress of the treatment of the abnormal portion by the focused ultrasound signal through the image regarding the target 1450. Specifically, the processor 120 may determine the progress of the treatment of the abnormal portion 1440 by the focused ultrasound signal, based on the image regarding the target 1450, and show the image to a user, such that the user can identify the progress of the treatment of the abnormal portion 1440 by the focused ultrasound signal.

Referring back to FIG. 13, the processor 120 may control the transmission/reception selection and group mapping unit 121 to group the plurality of cells included in the matrix array AFE 110 into a first group and a second group corresponding to the diagnosis target 1 1312 and the treatment target 1322, respectively.

Herein, the first group may correspond to the transmission and reception beamforming processor 1 1310 and a first region 1311 of the transducer 10, and the second group may correspond to the transmission and reception beamforming processor N+1 1320 and a second region 1321 of the transducer 10.

In addition, the first region 1311 of the transducer 10 may transmit and receive ultrasound signals to and from the diagnosis target 1 1312, and the second region 1321 of the transducer 10 may transmit, to the treatment target 1322, a focused ultrasound signal generated by the HIFU pulse generator 127.

In response to the ultrasound signal being received from the diagnosis target 1 1312, the received ultrasound signal may be transmitted to the B mode processor 124 through the transmission and reception beamforming processor 1 1310, and the B mode processor 124 may generate an image by processing the received ultrasound signal, and the Doppler mode processor 126 and the image synthesis unit 125 may generate a synthesis image related to the progress of the treatment by comparing and analyzing the generated plurality of images.

The pulse generator 122 may generate pulses to be used to output the ultrasound signals at the respective regions 1311, 1321 of the transducer 10.

As described above, the probe device 100 according to an embodiment of the present disclosure may group the plurality of cells included in the matrix array AFE 110 into the first group and the second group, and may output a focused ultrasound signal suitable for operation, such as an HIFU, through the first group, and may transmit and receive ultrasound signals through the second group and may obtain an image for guiding the operation or showing the progress of the operation.

Accordingly, compared to the related-art ultrasound examination system requiring two different probe devices to diagnose and treat, the probe device according to the present disclosure can implement functions for operation and diagnosis or guide of the operation simply by changing the configuration of the matrix array AFE 110 without adding hardware.

[Fourth Mode for Transmitting a High-Voltage/Low-Voltage Ultrasound Signal]

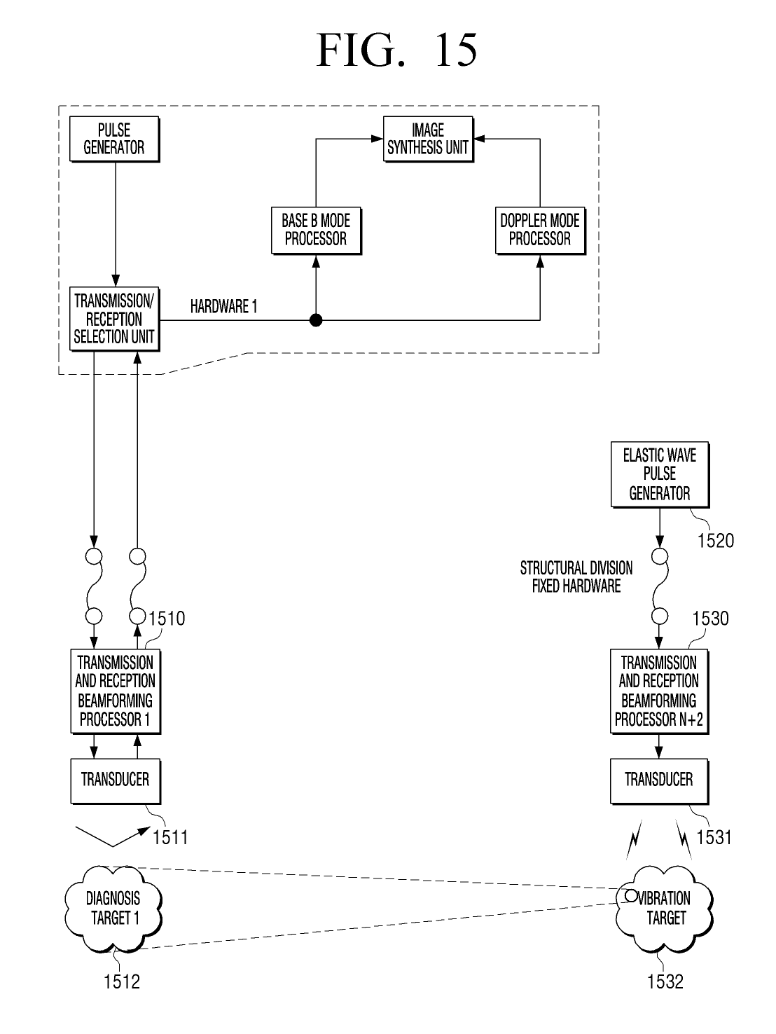

FIGS. 15 to 17 are views to illustrate a process of transmitting a high-voltage/low-voltage ultrasound signal according to an embodiment of the present disclosure. In particular, FIG. 15 illustrates a related-art technology used for transmitting a high-voltage/low-voltage ultrasound signal. Referring to FIG. 15, a related-art ultrasound examination system may separately include a probe device for transmitting and receiving ultrasound signals to and from a diagnosis target 1512, and a probe device for transmitting and receiving ultrasound signals to and from a vibration target 1532.

For example, the related-art ultrasound examination system separately includes a probe device including a transducer 1531 for transmitting a high-voltage ultrasound signal to the vibration target 1532, a transmission beamforming processor N+2 1530, an elastic wave pulse generator 1520, etc., and a probe device including a transducer 1511 for transmitting and receiving ultrasound signals to and from the diagnosis target 1 1512, a transmission and reception beamforming processor 1 1510, a pulse generator, a transmission/reception selection unit, a base B mode processor, an image synthesis unit, a Doppler mode processor, etc.

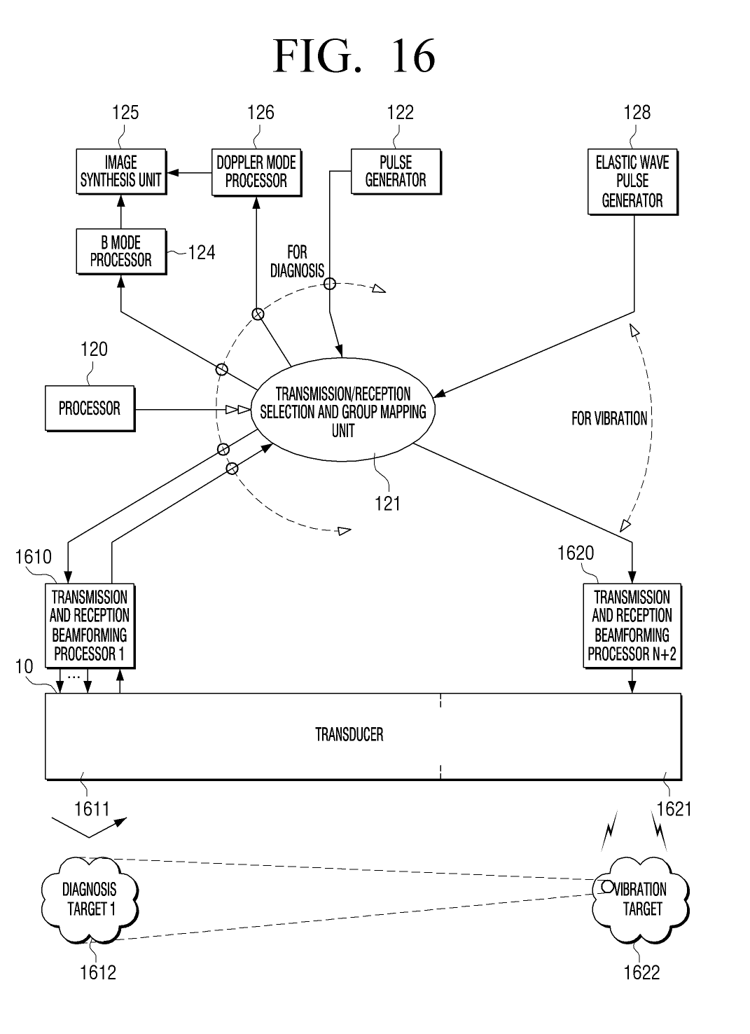

FIG. 16 illustrates a process of transmitting a high-voltage/low-voltage ultrasound signal according to an embodiment of the present disclosure. Referring to FIG. 16, the processor 120 may control a transmission and reception beamforming processor N+2 1620 through the transmission/reception selection and group dynamic mapping unit 121 to transmit a high-voltage ultrasound signal to a vibration target 1622, and may control a transmission and reception processor 1 1610 to transmit a low-voltage ultrasound signal to a diagnosis target 1 1612.

In particular, in the fourth mode for transmitting the high-voltage/low-voltage ultrasound signal, the processor 120 may divide a plurality of cells belonging to a group corresponding to the fourth mode into a first transmission and reception region to generate a high-voltage ultrasound signal and transmit and receive the high-voltage ultrasound signal, and a second transmission and reception region to generate a low-voltage ultrasound signal and transmit and receive the low-voltage ultrasound signal, and may generate a first image based on the transmitted and received high-voltage ultrasound signal, and may generate a second image based on the transmitted and received low-voltage ultrasound signal.

Herein, each of the transmitted and received high-voltage and low-voltage ultrasound signals may be used for generating an image, and may induce a change in elasticity of a measurement target tissue to generate an image.

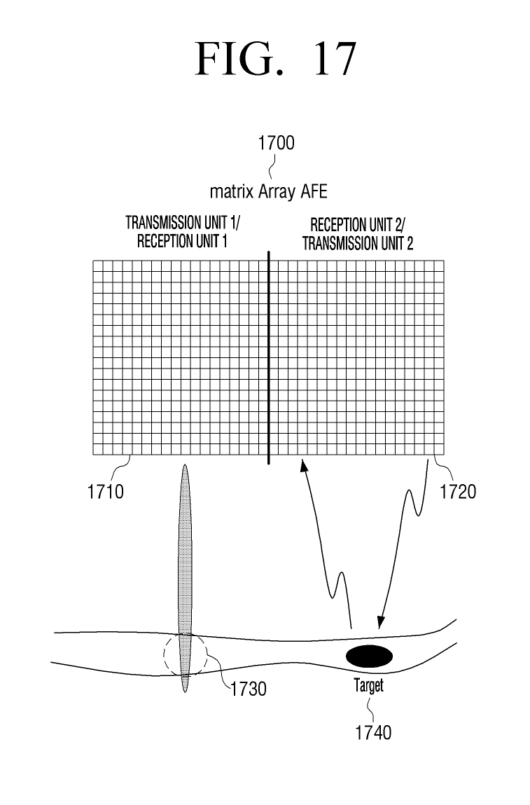

Specifically, referring to FIG. 17, when it is assumed that all of the plurality of cells included in a matrix array AFE 1700 are grouped into a group corresponding to the fourth mode for transmitting a high-voltage/low-voltage ultrasound signal, it can be known that the plurality of cells belonging to the group corresponding to the fourth mode are divided into a transmission unit 1/reception unit 1 1710 and a transmission unit 2/reception unit 2 1720.

In addition, the processor 120 may transmit a high-voltage ultrasound signal to a target 1730 through the transmission unit 1/reception unit 1 1710, and may transmit and receive low-voltage ultrasound signals to and from a target 1740 through the transmission unit 2/reception unit 2 1720.

Herein, the processor 120 may transmit a shear wave, that is, an S wave, to the target 1730 as an example of the high-voltage ultrasound signal. In response to the high-voltage ultrasound signal being transmitted to the target 1730, a motion may be generated on a region corresponding to the target 1730 by the high-voltage ultrasound signal. That is, an elastic motion occurs on the region corresponding to the target 1730 by the high-voltage ultrasound signal to generate an elastic wave image.

For example, referring to FIG. 17, the elastic motion generated on the region corresponding to the target 1730 by the high-voltage ultrasound signal may influence the target 1740, and accordingly, the processor 120 may obtain an elasticity image related to the target 1740. Specifically, the processor 120 may obtain the elasticity image related to the target 1740 by transmitting and receiving low-voltage ultrasound signals to and from the target 1740 through the transmission unit 2/reception unit 2 1720.

As described above, the processor 120 may generate the elastic wave image by using the high-voltage ultrasound signal, and also may use the high-voltage ultrasound signal for the purpose of a B-mode image. In addition, the processor 120 may use the low-voltage ultrasound signal in a Doppler mode for generating a plurality of images related to a plurality of targets.

Referring back to FIG. 16, the processor 120 may control the transmission/reception selection and group mapping unit 121 to group the plurality of cells included in the matrix array AFE 110 into a first group and a second group corresponding to the diagnosis target 1 1612 and the vibration target 1622, respectively.

Herein, the first group may correspond to the transmission and reception beamforming processor 1 1610 and a first region 1611 of the transducer 10, and the second group may correspond to the transmission and reception beamforming processor N+2 1620 and a second region 1621 of the transducer 10.

In addition, the second region 1621 of the transducer 10 may transmit a high-voltage ultrasound signal to the vibration target 1622, and the first region 1611 of the transducer 10 may transmit and receive low-voltage ultrasound signals to and from the diagnosis target 1 1612.

In response to the ultrasound signal being received from the diagnosis target 1 1612, which is influenced by the elastic motion generated at the vibration target 1622 by the high-voltage ultrasound signal, the received ultrasound signal may be transmitted to the B mode processor 124 through the transmission and reception beamforming processor 1 1610, and the B mode processor 124 may generate an elasticity image by processing the received ultrasound signal, and the Doppler mode processor 126 and the image synthesis unit 125 may generate a synthesis image related to an abnormal portion by comparing and analyzing the generated plurality of elasticity images.

As described above, the probe device 100 according to an embodiment of the present disclosure may group the plurality of cells included in the matrix array AFE 110 into the first group and the second group, and may output a high-voltage ultrasound signal for generating an elastic motion through the second group, and may transmit and receive low-voltage ultrasound signals through the first group and may generate an elasticity image according to the elastic motion.

Accordingly, compared to the related-art ultrasound examination system separately requiring both the probe device for transmitting and receiving high-voltage ultrasound signals, and the probe device for transmitting and receiving low-voltage ultrasound signals, the probe device 100 according to an embodiment of the present disclosure can output the high-voltage ultrasound signal and the low-voltage ultrasound signal simultaneously simply by changing the configuration of the matrix array AFE 110 without adding separate hardware.

The processor 120 may perform beamforming with respect to a plurality of cells belonging to each group based on at least one of a depth, a size, and a location of a target, such that an ultrasound signal outputted through the transducer unit 130 is focused onto the target.

For example, in the second mode for generating the plurality of images related to the plurality of targets described in FIG. 11, the depth, size, and location of the target 1 1150 and the depth, size, and location of the target 2 1160 may be different from each other.

Accordingly, the processor 120 may perform beamforming with respect to the first group corresponding to the first region from among the plurality of cells included in the matrix array AFE 110, based on the depth, size, and location of the target 1 1150, such that an ultrasound signal is focused onto the target 1 1150 through the first region of the transducer unit 130. That is, the processor 120 may control each cell of the first group to output an electric signal for allowing an ultrasound signal to be focused onto one target 1 1150.

In addition, the processor 120 may perform beamforming with respect to the second group corresponding to the second region from among the plurality of cells included in the matrix array AFE 110, based on the depth, size, and location of the target 2 1160, such that an ultrasound signal is focused onto the target 2 1160 through the second region of the transducer unit 130. That is, the processor 120 may control each cell of the second group to output an electric signal for allowing an ultrasound signal to be focused onto one target 2 1160.

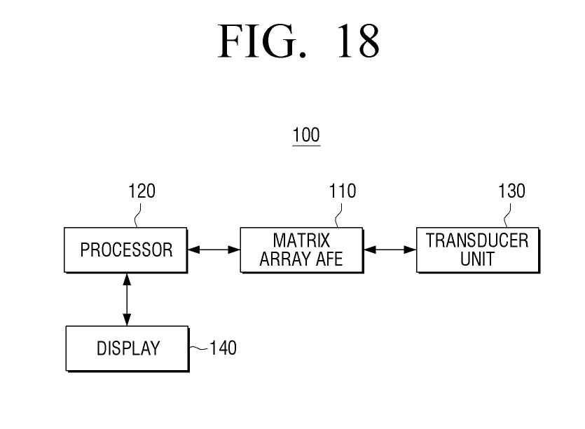

FIG. 18 is a block diagram showing a configuration of a probe device according to another embodiment of the present disclosure.

The probe device 100 according to an embodiment of the present disclosure may include a display 140 in addition to the matrix array AFE 110, the processor 120, and the transducer unit 130. Herein, since the matrix array AFE 110, the processor 120, and the transducer unit 130 have been already described, a detailed description thereof is omitted.

In addition, the processor 120 may display a generated image through the display 140.

Specifically, the processor 120 may display, through the display 140, a second harmonic image generated in the first mode, an image regarding an abnormal portion generated based on a plurality of images related to a plurality of targets in the second mode, an image regarding the progress of a treatment by a focused ultrasound signal in the third mode, and an elasticity image generated by a high-voltage ultrasound signal in the fourth mode.

In addition, the processor 120 may display, through the display 140, guide information regarding an examination or diagnosis and guide information regarding an operation or treatment in each mode.

Accordingly, the user can diagnose and treat more exactly by checking the guide information through the display 140 of the probe device 100.

That is, a related-art ultrasound examination system normally includes a device for displaying an image, and a probe device for examining a target in close contact with the target, whereas the probe device 100 according to an embodiment of the present disclosure includes the display 140 and thus performs both the function of examining and the function of displaying a generated image.

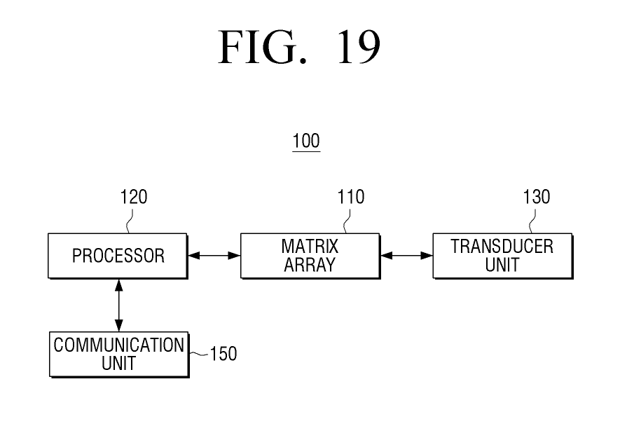

FIG. 19 is a block diagram showing a configuration of a probe device according to still another embodiment of the present disclosure.

The probe device 100 according to an embodiment of the present disclosure may include a communication unit 150 in addition to the matrix array AFE 110, the processor 120, and the transducer unit 130. Herein, since the matrix array AFE 110, the processor 120, and the transducer unit 130 have been already described, a detailed description thereof is omitted.

In addition, the processor 120 may control the communication unit to transmit a generated image to an external device and to display the image.

For example, the processor 120 may transmit a generated image to a portable terminal device such as a mobile device or a PDA, and may control to display the image through the portable terminal device.

The communication unit 150 may communicate with an external device in various communication methods. Herein, the communication unit 150 may communicate with at least one second electronic device through various communication methods such as Bluetooth (BT), wireless fidelity (Wi-Fi), Zigbee, infrared (IR), a serial interface, a universal serial bus (USB), near field communication (NFC), or the like.

In addition, the external device may be implemented by using various types of electronic devices such as TVs, electronic boards, electronic tables, large format display (LFDs), smart phones, tablets, desktop PCs, notebook PCs, servers, or the like.

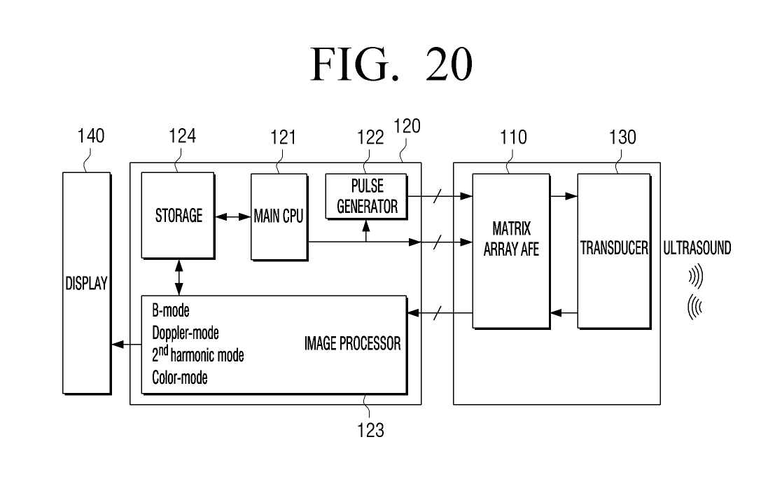

FIG. 20 is a block diagram showing a detailed configuration of the probe device 100 shown in FIG. 2.

Referring to FIG. 20, the probe device 100 may include the matrix array AFE 110, the processor 120, the transducer unit 130, and the display 140.

The processor 120 may control the overall operation of the probe device 100.

Specifically, the processor 120 includes a main central processing unit (CPU) 121, the pulse generator 122, an image processor 123, and a storage. Although not shown, the processor 120 may include first to n-th interfaces, and the first to n-th interfaces may be connected with the above-described various elements. One of the interfaces may be a network interface connected to an external device via a network.

The main CPU 121 may access the storage 124 and may perform booting using an operating system (O/S) stored in the storage 124. In addition, the main CPU 121 performs various operations by using various programs, contents, data, etc. stored in the storage 124.

In addition, the processor 120 may include a read only memory (ROM) (not shown), a random access memory (RAM) (not shown), etc. in addition to the storage 124. The ROM (not shown) stores a set of instructions for booting a system. In response to a turn-on command being inputted and power being supplied, the main CPU 121 copies the O/S stored in the storage 124 onto the RAM (not shown) according to the instruction stored in the ROM (not shown), executes the O/S and boots the system. In response to booting being completed, the main CPU 121 copies various application programs stored in the storage 124 onto the RAM (not shown), executes the programs copied onto the RAM (not shown), and performs various operations.

The image processor 123 may generate a screen including various objects such as an icon, an image, a text, and the like, by using a calculator (not shown) and a renderer (not shown). The calculator (not shown) calculates attribute values of the objects to be displayed, such as coordinate values, shape, size, color, and the like, according to the layout of the screen, based on a received control command. The renderer (not shown) generates screens of various layouts including the objects based on the attribute values calculated by the calculator (not shown). The screen generated by the renderer (not shown) may be displayed through the display 140.

In particular, the image processor 123 may generate images of B-mode, Doppler-mode, second harmonic mode, color-mode based on ultrasound signals received through the transducer unit 130.

The operations of the processor 120 described above with reference to FIGS. 1 to 19 may be performed by a program stored in the storage 124.

The storage 124 may store various data such as an O/S software module for driving the prove device 100 or various multimedia contents.

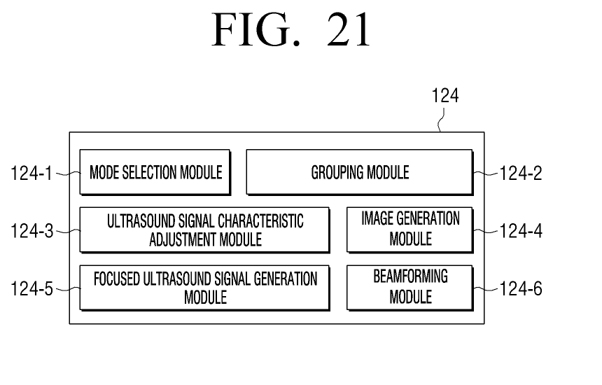

In particular, the storage 124 may include various software modules in order for the processor 120 to group the plurality of cells into at least one group corresponding to at least one diagnosis mode, and to control cells corresponding to each group to output an ultrasound signal having a different characteristic according to a corresponding diagnosis mode through the transducer unit 130. This will be described in detail with reference to FIG. 21.

The pulse generator 122 may generate a pulse for converting into an ultrasound signal, and may transmit the generated pulse to the matrix array AFE 110, and the matrix array AFE 110 may transmit the pulse to a corresponding region of the transducer unit 130 according to each group grouped by the processor 120 so as to transduce the pulse into an ultrasound signal.

FIG. 21 is a view showing a software module stored in the storage according to an embodiment of the present disclosure.

Referring to FIG. 21, the storage 124 may store programs including a mode selection module 124-1, a grouping module 124-2, an ultrasound signal characteristic adjustment module 124-3, an image generation module 124-4, a focused ultrasound signal generation module 124-5, and a beamforming module 124-6.

The above-described operations of the processor 120 may be performed by a program stored in the storage 124. Hereinafter, detailed operations of the processor 120 using the program stored in the storage 124 will be described.

The mode selection module 124-1 is a module for determining which mode of the above-described diagnosis modes, that is, the first mode for generating a second harmonic image, the second mode for generating a plurality of images related to a plurality of targets, a third mode for transmitting a focused ultrasound signal, and a fourth mode for transmitting a high-voltage/low-voltage ultrasound signal, is selected.

In addition, various diagnosis modes other than the first to fourth modes may be selected.

In addition, the grouping module 124-2 performs the function of grouping the plurality of cells included in the matrix array AFE 110 into a group corresponding to a mode selected by the mode selection module 124-1.

In addition, the ultrasound signal characteristic adjustment module 124-3 performs the function of adjusting at least one of a size, a frequency, and a focusing point of an ultrasound signal in cells corresponding to the diagnosis mode according to the selected diagnosis mode.

In addition, the image generation module 124-4 may generate an image corresponding to each diagnosis mode based on a received ultrasound signal.

In addition, the focused ultrasound signal generation module 124-5 may perform the function of controlling the HIFU pulse generator 127 to generate a focused ultrasound signal.

In addition, the beamforming module 124-6 may perform beamforming with respect to a plurality of cells belonging to each group based on at least one of a depth, a size, and a location of a target, such that an ultrasound signal outputted through the transducer unit 130 is focused onto the target.

The above-described diagnosis modes include the first mode for generating a second harmonic image, the second mode for generating a plurality of images related to a plurality of targets, the third mode for transmitting a focused ultrasound signal, and the fourth mode for transmitting a high-voltage/low-voltage ultrasound signal, and the first mode, the second mode, the third mode, and the fourth mode may correspond to a second harmonic mode, a Doppler mode, an HIFU mode, and an elastic wave mode, respectively.

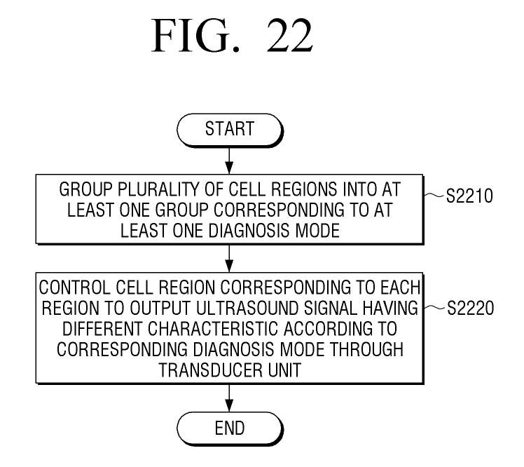

FIG. 22 is a flowchart to illustrate a control method of a probe device according to an embodiment of the present disclosure.

As shown in FIG. 22, a control method of a probe device including: a matrix array AFE including a plurality of cells, and outputting an electric signal corresponding to each of the plurality of cells, and a transducer unit to transduce the electric signal outputted from each of the plurality of cells into an ultrasound signal, may group the plurality of cells into at least one group corresponding to at least one diagnosis mode (S2210).

In addition, the control method may control cells corresponding to each group to output an ultrasound signal having a different characteristic according to a corresponding diagnosis mode through the transducer unit (S2220).

The control method of the probe device according to an embodiment of the present disclosure may further include transmitting the ultrasound signal having the different characteristic to a target, and generating a different image based on an ultrasound signal received from the target, or transmitting a focused ultrasound signal to the target.

Herein, the controlling may include controlling cells corresponding to each diagnosis mode to adjust at least one of a size, a frequency, and a focusing point of the ultrasound signal, and to output a different ultrasound signal.

In addition, the diagnosis mode may include a first mode for generating a second harmonic image, a second mode for generating a plurality of images related to a plurality of targets, a third mode for transmitting a focused ultrasound signal, and a fourth mode for transmitting a high-voltage/low-voltage ultrasound signal.

The control method of the probe device according to an embodiment of the present disclosure may further include, in the first mode, dividing a plurality of cells belonging to a group corresponding to the first mode into a first transmission and reception region and a second transmission and reception region, transmitting a first ultrasound signal and a second ultrasound signal having a phase difference of 180 degrees to a target, simultaneously, through the first transmission and reception region and the second transmission and reception region, and generating the second harmonic image based on the first ultrasound signal and the second ultrasound signal received from the target.

In addition, the control method of the probe device according to an embodiment of the present disclosure may further include, in the second mode, dividing a plurality of cells belonging to a group corresponding to the second mode into a plurality of transmission and reception regions to transmit and receive the ultrasound signal to and from the plurality of targets, and detecting an abnormal portion by comparing images generated from the plurality of transmission and reception regions.

In addition, the control method of the probe device according to an embodiment of the present disclosure may further include, in the third mode, dividing a plurality of cells belonging to a group corresponding to the third group into a first transmission region and a second transmission and reception region, transmitting a focused ultrasound signal for a treatment to a target through the first transmission region, transmitting and receiving the ultrasound signal to and from the target through the second transmission and reception region, and generating an image regarding a progress of a treatment by the focused ultrasound signal.

In addition, the control method of the probe device according to an embodiment of the present disclosure may further include, in the fourth mode, dividing a plurality of cells belonging to a group corresponding to the fourth mode into a first transmission and reception region for generating and transmitting and receiving a high-voltage ultrasound signal, and a second transmission and reception region for generating and transmitting and receiving a low-voltage ultrasound signal, generating a first image based on the transmitted and received high-voltage ultrasound signal, and generating a second image based on the transmitted and received low-voltage ultrasound signal.

In addition, the control method of the probe device according to an embodiment of the present disclosure may further include performing beamforming with respect to the plurality of cells belonging to each group, based on at least one of a depth, a size, and a location of a target, such that an ultrasound signal outputted through the transducer unit is focused onto the target.

In addition, the control method of the probe device according to an embodiment of the present disclosure may further include displaying the generated image.

In addition, the control method of the probe device according to an embodiment of the present disclosure may further include controlling to transmit the generated image to an external device and to display the image.

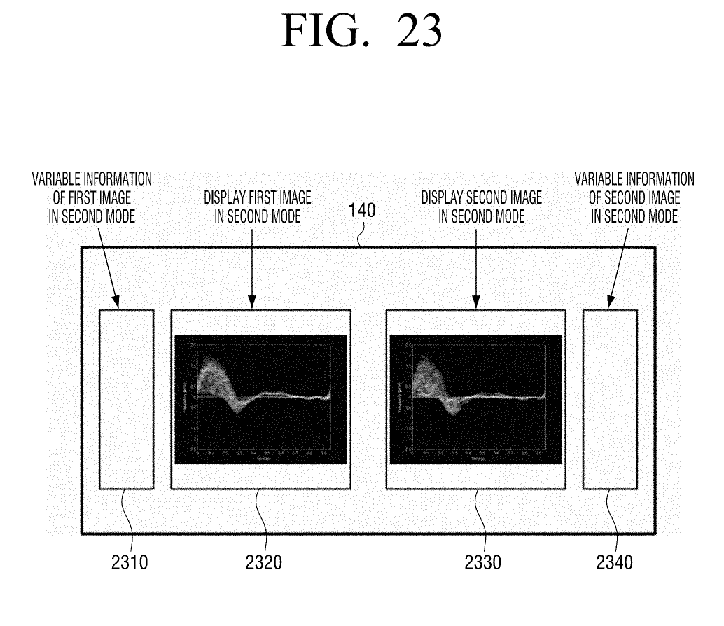

FIGS. 23 to 25 are views showing images generated according to respective modes according to an embodiment of the present disclosure.

Referring to FIG. 23, when the processor 120 operates in the second mode for generating a plurality of images related to a plurality of targets, a plurality of images 2320, 2330 regarding a plurality of targets may be displayed on the display 140.

In particular, a window 2310 for separately displaying variable information related to the first image 2320 currently displayed may be displayed on the left of the first image 2320 related to the first target, and the variable information may include information regarding a size, a frequency, and a focusing point of an ultrasound signal.

Likewise, a window 2340 for separately displaying variable information related to the second image 2330 currently displayed may be displayed on the right of the second image 2330 related to the second target, and the variable information may include information regarding a size, a frequency, and a focusing point of an ultrasound signal.

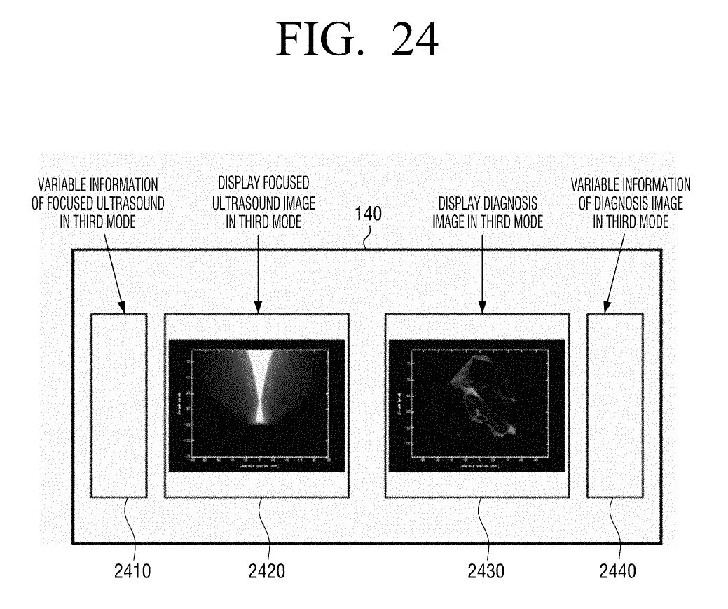

Referring to FIG. 24, when the processor 120 operates in the third mode for transmitting a focused ultrasound signal, a focused ultrasound image 2420 and a diagnosis image 2430 regarding a target to which focused ultrasound is applied may be displayed on the display 140.

In particular, a window 2410 for separately displaying variable information of the focused ultrasound related to the focused ultrasound image 2420 currently displayed may be displayed on the left of the focused ultrasound image 2420, and the variable information may include information regarding a size, a frequency, and a focusing point of the focused ultrasound.

Likewise, a window 2440 for separately displaying variable information related to the diagnosis image 2430 currently displayed may be displayed on the right of the diagnosis image 2430 regarding the target to which the focused ultrasound is applied, and the variable information may include information regarding a size and a frequency of the focused ultrasound, and a location and a depth of the target.

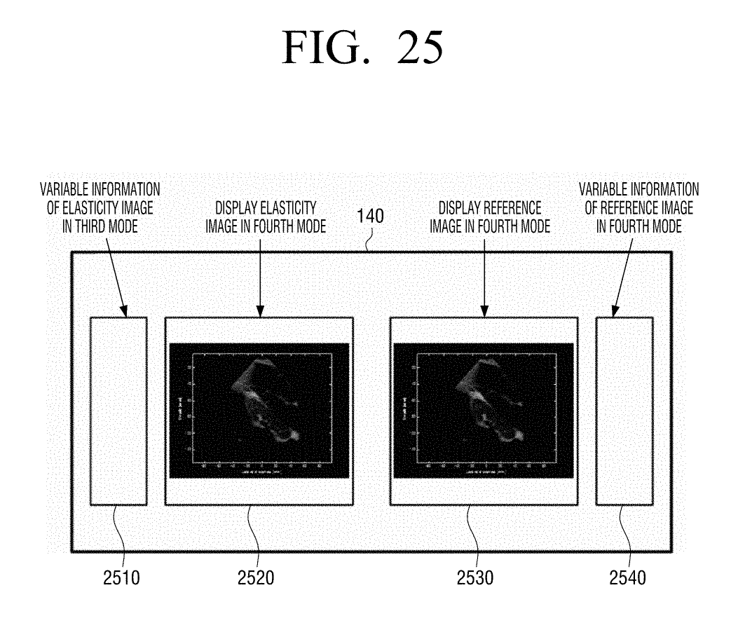

Referring to FIG. 25, when the processor 120 operates in the fourth mode for transmitting a high-voltage/low-voltage ultrasound signal, an elasticity image 2520 and a reference image 2530 may be displayed on the display 140. Herein, the reference image 2530 may refer to an image before a high-voltage ultrasound signal is applied, that is, when elasticity is not applied.

In particular, a window 2510 for separately displaying variable information of the high-voltage ultrasound related to the elasticity image 2520 currently displayed may be displayed on the left of the elasticity image 2520, and the variable information may include information regarding a size, a frequency, and a focusing point of the high-voltage ultrasound.

Likewise, a window 2540 for separately displaying variable information related to the reference image 2530 currently displayed may be displayed on the right of the reference image 2530, and the variable information may include information regarding a size, a frequency, and a focusing point of ultrasound used for obtaining the reference image 2530.

The control method of the probe device according to various embodiments of the present disclosure described above may be implemented as a program code executable by a computer, and may be stored in various non-transitory computer readable media, and may be provided to each device to be executed by a controller.

For example, there is provided a non-transitory computer readable medium which stores a program performing a control method, including the steps of: grouping a plurality of cells into at least one group corresponding to at least one diagnosis mode; and controlling cells corresponding to each group to output an ultrasound signal having a different characteristic according to a corresponding diagnosis mode through a transducer unit.

The non-transitory computer readable medium refers to a medium that stores data semi-permanently rather than storing data for a very short time, such as a register, a cache, a memory or etc., and is readable by an apparatus. Specifically, the above-described various applications or programs may be stored in the non-transitory computer readable medium such as a compact disc (CD), a digital versatile disk (DVD), a hard disk, a Blu-ray disk, a universal serial bus (USB), a memory card, a ROM or etc., and may be provided.

Although a bus is not illustrated in the above-described block diagrams of the probe device, communication among the respective elements in the probe device may be performed through a bus. In addition, each device may further include a controller such as a CPU, a micro controller, or the like to perform the above-described various steps.

While preferred embodiments of the present disclosure have been illustrated and described, the present disclosure is not limited to the above-described specific embodiments. Various changes can be made by a person skilled in the art without departing from the scope of the present disclosure claimed in claims, and also, changed embodiments should not be understood as being separate from the technical idea or prospect of the present disclosure.

What is claimed is:

1. A probe device comprising:
a matrix array analog front end (AFE) comprising a plurality of cells, and configured to output an electric signal corresponding to each of the plurality of cells;
a transducer unit configured to transduce the electric signal outputted from each of the plurality of cells into an ultrasound signal; and
a processor configured to group the plurality of cells into at least one group corresponding to at least one diagnosis mode, and to control cells corresponding to each group to output an ultrasound signal having a different characteristic according to a corresponding diagnosis mode through the transducer unit.

2. The probe device of claim 1, wherein the processor is configured to transmit the ultrasound signal having the different characteristic to a target, and to generate a different image based on an ultrasound signal received from the target, or to transmit a focused ultrasound signal to the target.

3. The probe device of claim 1, wherein the processor is configured to control cells corresponding to each diagnosis mode to adjust at least one of a size, a frequency, and a focusing point of the ultrasound signal, and to output a different ultrasound signal.

4. The probe device of claim 1, wherein the diagnosis mode comprises a first mode for generating a second harmonic image, a second mode for generating a plurality of images related to a plurality of targets, a third mode for transmitting a focused ultrasound signal, and a fourth mode for transmitting a high-voltage/low-voltage ultrasound signal.

5. The probe device of claim 4, wherein, in the first mode, the processor is configured to divide a plurality of cells belonging to a group corresponding to the first mode into a first transmission and reception region and a second transmission and reception region, to transmit a first ultrasound signal and a second ultrasound signal having a phase difference of 180 degrees to a target, simultaneously, through the first transmission and reception region and the second transmission and reception region, and to generate the second harmonic image based on the first ultrasound signal and the second ultrasound signal received from the target.

6. The probe device of claim 4, wherein, in the second mode, the processor is configured to divide a plurality of cells belonging to a group corresponding to the second mode into a plurality of transmission and reception regions to transmit and receive the ultrasound signal to and from the plurality of targets, and to detect an abnormal portion by comparing images generated from the plurality of transmission and reception regions.

7. The probe device of claim 4, wherein, in the third mode, the processor is configured to divide a plurality of cells belonging to a group corresponding to the third group into a first transmission region and a second transmission and reception region, to transmit a focused ultrasound signal for a treatment to a target through the first transmission region, to transmit and receive the ultrasound signal to and from the target through the second transmission and reception region, and to generate an image regarding a progress of a treatment by the focused ultrasound signal.

8. The probe device of claim 4, wherein, in the fourth mode, the processor is configured to divide a plurality of cells belonging to a group corresponding to the fourth mode into a first transmission and reception region for generating and transmitting and receiving a high-voltage ultrasound signal, and a second transmission and reception region for generating and transmitting and receiving a low-voltage ultrasound signal, to generate a first image based on the transmitted and received high-voltage ultrasound signal, and to generate a second image based on the transmitted and received low-voltage ultrasound signal.

9. The probe device of claim 1, wherein the processor is configured to perform beamforming with respect to the plurality of cells belonging to each group, based on at least one of a depth, a size, and a location of a target, such that an ultrasound signal outputted through the transducer unit is focused onto the target.

10. The probe device of claim 2, further comprising a display,
wherein the processor is configured to display the generated image through the display.

11. The probe device of claim 2, further comprising a communication unit configured to communicate with an external device,
wherein the processor is configured to control the communication unit to transmit the generated image to the external device and to display the image.

12. A control method of a probe device comprising: a matrix array AFE comprising a plurality of cells, and configured to output an electric signal corresponding to each of the plurality of cells; and a transducer unit configured to transduce the electric signal outputted from each of the plurality of cells into an ultrasound signal, the control method comprising:
grouping the plurality of cells into at least one group corresponding to at least one diagnosis mode; and
controlling cells corresponding to each group to output an ultrasound signal having a different characteristic according to a corresponding diagnosis mode through the transducer unit.

13. The control method of claim 12, further comprising transmitting the ultrasound signal having the different characteristic to a target, and generating a different image based on an ultrasound signal received from the target, or transmitting a focused ultrasound signal to the target.

14. The control method of claim 12, wherein the controlling comprises controlling cells corresponding to each diagnosis mode to adjust at least one of a size, a frequency, and a focusing point of the ultrasound signal, and to output a different ultrasound signal.

15. The control method of claim 12, wherein the diagnosis mode comprises a first mode for generating a second harmonic image, a second mode for generating a plurality of images related to a plurality of targets, a third mode for transmitting a focused ultrasound signal, and a fourth mode for transmitting a high-voltage/low-voltage ultrasound signal.
